(19) United States
(12) Patent Application Publication
(10) Pub. No.: US 2022/0064640 A1
(43) Pub. Date: Mar. 3, 2022

(54) DOUBLE-STRANDED NUCLEIC ACID INHIBITOR MOLECULES CONTAINING A TRILOOP

(71) Applicant: Dicerna Pharmaceuticals, Inc., Lexington, MA (US)

(72) Inventor: Bob Dale BROWN, Littleton, MA (US)

(21) Appl. No.: 17/311,949

(22) PCT Filed: Nov. 13, 2019
(86) PCT No.: PCT/US2019/061241

§ 371 Date: Jun. 8, 2021

Related U.S. Application Data
(60) Provisional application No. 62/778,759, filed on Dec. 12, 2018.

(51) Int. Cl. C12N 15/113; A61K 47/54

(52) U.S. Cl. CPC C12N 15/113; A61K 47/549; C12N 2310/14; C12N 2310/351; C12N 2320/30; C12N 2310/3231

(57) ABSTRACT

Provided herein are double-stranded nucleic acid inhibitor molecules having a sense strand with a stem loop structure and an antisense strand, where the loop portion of the stem loop structure is a triloop. Also provided are methods and compositions for reducing target gene expression and methods and compositions for treating a disease of interest.

This application claims the benefit of, and relies on the filing date of, U.S. provisional patent application No. 62/778,759, filed 12 Dec. 2018, the entire contents of which are incorporated herein by reference.

### BACKGROUND

Oligonucleotides are polymeric sequences of nucleotides (RNA, DNA and their analogs). Nucleic acid inhibitor molecules are oligonucleotides that modulate intracellular RNA levels and have demonstrated early promise in the treatment of cancers, viral infections and genetic disorders. Nucleic acid inhibitor molecules can modulate RNA expression through a diverse set of mechanisms, including RNA interference (RNAi).

RNAi is a conserved pathway found in most eukaryotes where double-stranded RNA molecules (dsRNA) inhibit the expression of target genes having sequences complementary to the dsRNA. In the typical RNAi pathway, a longer dsRNA molecule is cleaved by the Dicer enzyme into shorter RNA duplexes called small interfering RNAs (“siRNA”). The siRNA has been shown to associate with Dicer, trans-activating response RNA-binding protein (TRBP), and Argonaute 2 (“Ago2”) to form a complex, sometimes referred to as the RNA-induced silencing complex (“RISC”). Ago2 once activated is an endonuclease that cleaves target mRNA using the antisense strand (also called the guide strand) of the siRNA to direct the sequence specificity of the RISC complex towards cleavage of the target mRNA.

A variety of double-stranded RNAi inhibitor molecule structures have been developed over the years. For example, early work on RNAi inhibitor molecules focused on double-stranded nucleic acid molecules that mimic natural siRNAs, with each strand having sizes of 19-25 nucleotides with at least one 3′-overhang of 1 to 5 nucleotides (see, e.g., U.S. Pat. No. 8,372,968). Subsequently, longer double-stranded RNAi inhibitor molecules that get processed in vivo by the Dicer enzyme to active RNAi inhibitor molecules were developed (see, e.g., U.S. Pat. No. 8,883,996). Later work developed extended double-stranded nucleic acid inhibitor molecules where at least one end of at least one strand is extended beyond the double-stranded targeting region of the molecule, including structures where one of the strands includes a thermodynamically-stabilizing tetraloop structure (see, e.g., U.S. Pat. Nos. 8,513,207, 8,927,705, WO 2010/033225, and WO 2016/100401, each of which is hereby incorporated by reference in its entirety).

In certain instances, chemically modified nucleotides have been introduced into nucleic acid inhibitor molecules to introduce properties that may be desired under specific conditions, such as conditions experienced following in vivo administration. Such chemically modified nucleotides include those designed, for example, to stabilize against nucleases or other enzymes that degrade or interfere with the structure or activity of the oligonucleotide, to increase cellular uptake of the oligonucleotide, or to improve the pharmacokinetic properties of the oligonucleotide.

However, the desire to develop new double-stranded nucleic acid inhibitor molecules and/or incorporate chemically modified nucleotides to impart desired properties to such nucleic acid inhibitor molecules must be balanced with the competing desire to minimize any negative impact that the structure and/or chemically modified nucleotides might have on the nucleic acid inhibitor molecule's activity (e.g., minimizing any reduction in the potency or duration of gene target knock down).

### SUMMARY

Disclosed herein are double-stranded nucleic acid inhibitor molecules having a sense strand with a stem loop structure and a separate antisense strand, where the loop portion of the stem loop structure contains a triloop. As shown in the examples, double-stranded nucleic acid inhibitor containing a triloop are stable and reduce in vivo target mRNA expression in a dose-dependent manner. It was surprising that this triloop structure, when removed from its naturally occurring context and incorporated into a chemically synthesized, double-stranded nucleic acid inhibitor molecule, was able to maintain a thermodynamically stable configuration. It was also surprising to find that conjugating a ligand, such as GalNAc, to the nucleotides in the triloop did not disrupt the thermodynamically stable configuration of the triloop and that conjugating 2 GalNAcs to the triloop conferred no reduction in potency in hepatocytes as compared to a tetraloop-containing double-stranded nucleic acid inhibitor molecule having 3 GalNAcs conjugated to the tetraloop and, in certain instances, actually improved potency.

Additionally, triloop-containing double-stranded nucleic acid inhibitor molecules may incorporate bicyclic nucleotides into the stem portion of the stem loop structure. As previously demonstrated in International Publication No. WO 2019/200124, incorporating Tₘ-increasing nucleotides into the stem duplex may impart increased stability to tetraloop-containing double-stranded nucleic acid inhibitor molecules, as evidenced, in part, by enhanced duration of in vivo target mRNA knock down.

Further, the use of a triloop in place of a tetraloop and the incorporation of bicyclic nucleotides into the stem portion of the stem loop structure permits the use of shorter sense strands without reducing potency of the double-stranded nucleic inhibitor molecules comprising the same. The use of shorter sense strands confers advantages in the manufacturing process, reducing both time and cost. It also confers advantages in dosing because it is possible to administer more of the triloop-containing double-stranded nucleic acid inhibitor molecule on a molar basis due to its reduced molecular weight.

The double-stranded nucleic acid inhibitor molecule contains a first duplex (D1) between a first region (R1) of the sense strand (S) and the antisense strand (AS) and a second duplex (D2) between a first subregion (S1) and a second subregion (S2) of a second region (R2) of the sense strand, where 51 and S2 are joined by the triloop (triL). See FIGS. 1A-D. Additionally, the stem portion of the stem loop structure may, in certain embodiments, contain at least one Tₘ-increasing nucleotide, such as from 4-12 Tₘ-increasing nucleotides, e.g., from 2-6 Tₘ-increasing nucleotide base pairs, or from 1-6 unpaired Tₘ-increasing nucleotides. The stem loop structure may be located at the 5′- or 3′-end of the sense strand.

In certain embodiments, the double-stranded nucleic acid inhibitor molecule, comprises:
a sense strand comprising 20-65 nucleotides and having a first region (R1) and a second region (R2);
an antisense strand comprising 15-40 nucleotides, wherein the sense strand and antisense strand are separate strands;
a first duplex (D1) formed by the first region of the sense strand and the antisense strand, wherein the first duplex has a length of 15-40 base pairs;
wherein the second region (R2) of the sense strand comprises a first subregion (S1), a second subregion (S2) and a triloop (triL) that joins the first and second regions, wherein the first and second regions form a second duplex (D2).

In certain embodiments, the triloop has a nucleotide sequence of GAA

In certain embodiments, the sense strand has 22-65 nucleotides. In certain embodiments, the sense strand has 25-39 nucleotides. In certain embodiments, the sense strand has 27-35 nucleotides.

In certain embodiments, the antisense strand has 20-24 nucleotides. In certain embodiments, the antisense strand has 20-22 nucleotides.

In certain embodiments, the nucleotide immediately adjacent to the 5′-end of the triloop is a C, and the nucleotide immediately adjacent to the 3′-end of the triloop is a G.

In certain embodiments, the antisense strand has a single stranded overhang of 1-4 nucleotides at its 3′-end. In certain embodiments, the single stranded overhang is 2 nucleotides in length.

In certain embodiments, the first duplex (D1) has a length of 18-30 base pairs. In certain embodiments, the first duplex (D1) has a length of 18-24 base pairs. In certain embodiments, the first duplex (D1) has a length of 20-22 base pairs.

In certain embodiments, the second duplex (D2) has a length of 2-6 base pairs. In certain embodiments, the second duplex does not contain any Tₘ-increasing nucleotides, and in certain embodiments the second duplex contains 4-10 Tₘ-increasing nucleotides and has a length of 2-5 base pairs.

In certain embodiments, the sense strand is between 25-39 nucleotides in length, the antisense strand is between 20-24 nucleotides in length, the first duplex has a length of 18-24 nucleotides, and the second duplex has a length of 2-6 base pairs. In certain embodiments, the sense strand is between 27-35 nucleotides in length, the antisense strand is between 20-22 nucleotides in length, the first duplex has a length of 18-22 base pairs, and the second duplex has a length of 2-3 base pairs.

In certain embodiments, the second duplex (D2) has a length of 2 base pairs. In certain embodiments, the second duplex (D2) has a length of 3 base pairs.

In certain embodiments, the first region of the sense strand (R1) is 20 nucleotides in length and the second region of the sense strand (R2) is 7-9 nucleotides in length;

wherein the first duplex (D1) formed by the first region of the sense strand and the antisense strand has a length of 20 base pairs;

wherein the second duplex (D2) formed by a first subregion (S1) and a subregion (S2) of the second region of the sense strand (R2) has a length of 2 or 3 base pairs and wherein the second duplex contains at least one Tₘ-increasing nucleotide;

wherein the antisense strand is 22 nucleotides in length and has a single-stranded overhang of two nucleotides at its 3′-end; and

wherein the triloop has a nucleotide sequence of GAA. In certain embodiments, R2 is 7 nucleotides in length and D2 has a length of 2 base pairs. In certain embodiments, R2 is 9 nucleotides in length and D2 has a length of 3 base pairs.

In certain embodiments, each nucleotide in the second duplex (D2) is a Tₘ-increasing nucleotide. In certain embodiments, the triloop-containing double-stranded nucleic acid inhibitor molecule does not contain any Tₘ-increasing nucleotides outside of the second duplex (D2).

In certain embodiments, the Tₘ-increasing nucleotide is selected from the group consisting of a bicyclic nucleotide, a tricyclic nucleotide, a G-clamp and analogues thereof, a hexitol nucleotide, and a modified nucleotide, wherein the modified nucleotide is not modified at the 2′-carbon of the sugar moiety with a 2′-F or a 2′-OMe. In certain embodiments the modified nucleotide is a 5-bromo-uracil, a 5-iodo-uracil, 5-propynyl-modified pyrimidine, a 2-amino adenine, a 2-thio uridine, 5 Me-thio uridine, or a pseudo uridine.

In certain embodiments wherein the triloop-containing double-stranded nucleic acid inhibitor molecule comprises at least one bicyclic nucleotide, the at least one bicyclic nucleotide has the structure of Formula I, II, III, IV, Va, or Vb. In certain embodiments, the at least one bicyclic nucleotide has the structure of one or more of Formula Ia, Ib, Ic, Id, Ie, or If. In certain embodiments, the at least one bicyclic nucleotide has the structure of one or more of Formula IIa, IIb, IIc, or IId. In certain embodiments, the at least one bicyclic nucleotide has the structure of Formula IIIa and/or IIIb. In certain embodiments, the at least one bicyclic nucleotide has the structure of Formula IVa and/or IVb.

In certain embodiments, the at least one bicyclic nucleotide is one or more of the following:

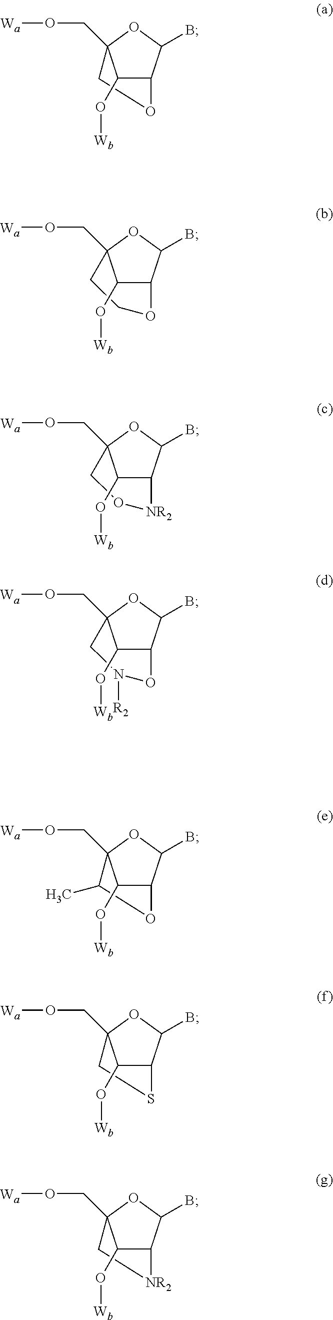

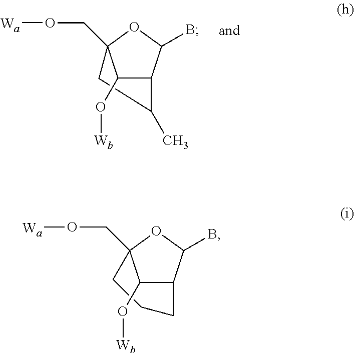

wherein B is a nucleobase, R2 is H or CH₃ and Wₐ and W_{b} are each independently, H, OH, a hydroxyl protecting group, a phosphorous moiety, or an internucleotide linking group attaching the bicyclic nucleotide to another nucleotide or to an oligonucleotide and wherein at least one of Wₐ or W_{b} is an internucleotide linking group attaching the bicyclic nucleotide to an oligonucleotide.

In certain embodiments, the at least one bicyclic nucleotide is:

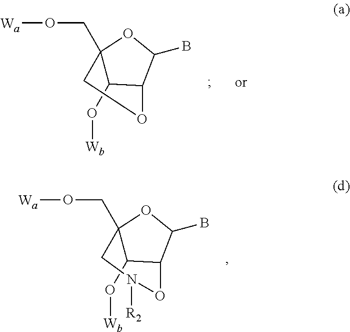

wherein B, Wₐ, and W_{b} are as described above and R₂ is CH₃.

In certain embodiments, the at least one bicyclic nucleotide comprises a first ring, wherein the first ring is a furanosyl, and a bridge that connects the 2′-carbon and the 4′-carbon of the furanosyl to form a second ring.

In certain embodiments, the bridge that connects the 2′-carbon and the 4′-carbon of the furanosyl is selected from the group consisting of:
a) 4′-CH₂—O—N(R)-2′ and 4′-CH₂—N(R)—O-2′, wherein R is H, C₁-C₁₂ alkyl, or a protecting group, including, for example, 4′-CH₂—NH—O-2′ (also known as BNA^{NC}) or 4′-CH₂—N(CH₃)—O-2′ (also known as BNA^{NC}[NMe]);
b) 4′-CH₂-2; 4′—(CH₂)₂-2; 4′—(CH₂)₃-2; 4′—(CH₂)—O-2′ (also known as LNA); 4′-(CH₂)—S-2; 4′—(CH₂)₂—O-2′ (also known as ENA); 4′-CH(CH₃)—O-2′ (also known as cEt); and 4′-CH(CH₂OCH₃)—O-2′ (also known as cMOE), and analogs thereof;
c) 4′-C(CH₃)(CH₃)—O-2′ and analogs thereof;
d) 4′-CH₂—N(OCH₃)-2′ and analogs thereof;
e) 4′-CH₂—O—N(CH₃)-2′ and analogs thereof;
f) 4′-CH₂—C(H)(CH₃)-2′ and analogs thereof; and
g) 4′-CH₂—C(═CH₂)-2′ and analogs thereof.

In certain embodiments, the triloop-containing double-stranded nucleic acid inhibitor molecule does not contain any Tₘ-increasing nucleotides outside of the second duplex.

In certain embodiments, the triloop comprises at least one ligand conjugated nucleotide. In certain embodiments, the triloop comprises two ligand conjugated nucleotides. In certain embodiments, the triloop comprises three ligand conjugated nucleotides. In certain embodiments, the ligand is a GalNAc. In certain embodiments, the GalNAc is conjugated to the nucleotide at the 2′-position of the sugar moiety.

In certain embodiments, the triloop-containing double-stranded nucleic acid inhibitor molecule further comprises a 5′-phosphate mimic at the 5′-terminus of the sense strand and/or the antisense strand.

In certain embodiments, the triloop-containing double-stranded nucleic acid inhibitor molecule is formulated with a lipid nanoparticle. In certain embodiments, the lipid nanoparticle comprises core lipids and envelope lipids, wherein the core lipids comprise a first cationic lipid and a first pegylated lipid and wherein the envelope lipids comprise a second cationic lipid, a neutral lipid, a sterol, and a second pegylated lipid. In certain embodiments, the first cationic lipid is DL-048, the first pegylated lipid is DSG-mPEG, the second cationic lipid is DL-103, the neutral lipid is DSPC, the sterol is cholesterol, and the second pegylated lipid is DSPE-mPEG.

Another aspect is directed to a pharmaceutical composition comprising a therapeutically effective amount of the triloop-containing double-stranded nucleic acid inhibitor molecule as described herein and a pharmaceutically acceptable excipient.

Another aspect is directed to a method for reducing expression of a target gene in a subject comprising administering the triloop-containing double-stranded nucleic acid inhibitor molecule or pharmaceutical composition to a subject in need thereof in an amount sufficient to reduce expression of the target gene. In certain embodiments, the administering step comprises intravenous, intramuscular, or subcutaneous administration. In certain embodiments, the subject is a human.

### BRIEF DESCRIPTION OF THE DRAWINGS

The accompanying drawings, which are incorporated in and constitute a part of this specification, illustrate certain embodiments, and together with the written description, serve to explain certain principles of the compositions and methods disclosed herein.

FIG. 1A shows a schematic of an exemplary double-stranded nucleic acid inhibitor molecule with an antisense strand (“AS”) and a sense strand (“S”), where the sense strand contains a stem loop structure and where the loop is a triloop.

FIG. 1B shows the same exemplary schematic as in FIG. 1A. In FIG. 1B, the sense strand is further divided into a first region (R1) that forms a duplex with the antisense strand (AS) and a second region (R2) that includes a triloop (triL) that joins a first subregion (S1) with a second subregion (S2), where S1 and S2 are sufficiently complementary to each other to form a duplex, referred to herein as a “stem” or “stem duplex.”

FIG. 1C schematically shows the same exemplary schematic as in FIGS. 1A and 1B. The schematic of FIG. 1C depicts a first duplex (D1) and a second duplex (D2) in the nucleic acid inhibitor molecule. The first duplex (D1) forms between the first region of the sense strand (R1) and the antisense strand (AS). The second duplex (D2) or “stem” forms between a first subregion (S1) and a second subregion (S2) of the second region (R2) of the sense strand.

FIG. 1D schematically shows an exemplary double-stranded nucleic acid inhibitor molecule where the second duplex (D2) is shorter than the second duplex depicted in FIG. 1C.

FIG. 2A schematically shows the structure of an exemplary double-stranded nucleic acid inhibitor molecule (“Construct 1”) that targets a gene sequence of interest, as discussed in Example 1. The sense strand of Construct 1 includes a stem duplex of 6 base pairs and a tetraloop. Three of the four nucleotides of the tetraloop are conjugated to a single GalNAc molecule.

FIG. 2B schematically shows the structure of an exemplary double-stranded nucleic acid inhibitor molecule (“Construct 2”) that targets a gene sequence of interest, as discussed in Example 1. The sense strand of Construct 2 includes a stem duplex of 6 base pairs and a tetraloop. The structure of Construct 2 is identical to the structure of Construct 1 except that only two of the four nucleotides of the tetraloop are conjugated to a single GalNAc molecule, instead of three of four, as in Construct 1.

FIG. 2C schematically shows the structure of an exemplary double-stranded nucleic acid inhibitor molecule (“Construct 3”) that targets a gene sequence of interest, as discussed in Example 1. The sense strand of Construct 3 includes a stem duplex of 6 base pairs and a triloop. Two of the three nucleotides of the triloop are conjugated to a single GalNAc molecule. The structure of Construct 3 is identical to the structure of Construct 2 except that the loop portion of the stem loop is a triloop instead of a tetraloop.

FIG. 2D schematically shows the structure of an exemplary double-stranded nucleic acid inhibitor molecule (“Construct 4”) that targets a gene sequence of interest, as discussed in Example 1. The sense strand of Construct 4 includes a stem duplex of 3 base pairs, wherein each of the nucleotides in the stem duplex is a bicyclic nucleotide, and a triloop. Two of the three nucleotides of the triloop are conjugated to a single GalNAc molecule.

FIG. 2E schematically shows the structure of an exemplary double-stranded nucleic acid inhibitor molecule (“Construct 5”) that targets a gene sequence of interest, as discussed in Example 1. The sense strand of Construct 5 includes a stem duplex of 3 base pairs, wherein each of the nucleotides in the stem duplex is an LNA, and a triloop. Two of the three nucleotides of the triloop are conjugated to a single GalNAc molecule.

FIG. 3A is a graph showing the percent of target gene mRNA remaining 4 days after administering to CD-1 mice varying dosages of Construct 1 (see FIG. 2A) and as explained in Example 1. As shown in FIG. 3A, the effective dose (ED₅₀) was calculated to be 0.5726 mg/kg.

FIG. 3B is a graph showing the percent of target gene mRNA remaining 4 days after administering to CD-1 mice varying dosages of Construct 2 (see FIG. 2B) and as explained in Example 1. As shown in FIG. 3B, the ED₅₀ was calculated to be 0.3604 mg/kg.

FIG. 3C is a graph showing the percent of target gene mRNA remaining 4 days after administering to CD-1 mice varying dosages of Construct 3 (see FIG. 2C) and as explained in Example 1. As shown in FIG. 3C, the ED₅₀ was calculated to be 0.3144 mg/kg.

FIG. 3D is a graph showing the percent of target gene mRNA remaining 4 days after administering to CD-1 mice varying dosages of Construct 4 (see FIG. 2D) and as explained in Example 1. As shown in FIG. 3D, the ED₅₀ was calculated to be 0.3012 mg/kg.

FIG. 3E is a graph showing the percent of target gene mRNA remaining 4 days after administering to CD-1 mice varying dosages of Construct 5 (see FIG. 2E) and as explained in Example 1. As shown in FIG. 3E, the ED₅₀ was calculated to be 0.2325 mg/kg.

FIG. 4 is a graph showing the overlap of percent of target gene mRNA remaining 4 days after administering to CD-1 mice varying dosages of Constructs 1-5 (see FIGS. 2A-E) and as explained in Example 1.

FIG. 5 is a bar graph showing the percent of target gene mRNA remaining 4 days after administering to CD-1 mice varying dosages of Constructs 1-5 (see FIGS. 2A-E) and as explained in Example 1.

FIG. 6A schematically shows the structure of an exemplary double-stranded nucleic acid inhibitor molecule (“Construct 6”) that targets a gene sequence of interest, as discussed in Example 2. The sense strand of Construct 6 includes a stem duplex of 6 base pairs and a tetraloop. Two of the four nucleotides of the tetraloop are conjugated to a single GalNAc molecule.

FIG. 6B schematically shows the structure of an exemplary double-stranded nucleic acid inhibitor molecule (“Construct 7”) that targets a gene sequence of interest, as discussed in Example 2. The sense strand of Construct 7 includes a stem duplex of 6 base pairs and a triloop. The structure of Construct 7 is identical to the structure of Construct 6, except that Construct 7 contains a triloop instead of a tetraloop.

FIG. 6C schematically shows the structure of an exemplary double-stranded nucleic acid inhibitor molecule (“Construct 8”) that targets a gene sequence of interest, as discussed in Example 2. The sense strand of Construct 8 includes a stem duplex of 3 base pairs, wherein each nucleotide in the stem portion of the stem loop structure is a BNA, and a tetraloop.

FIG. 6D schematically shows the structure of an exemplary double-stranded nucleic acid inhibitor molecule (“Construct 9”) that targets a gene sequence of interest, as discussed in Example 2. The sense strand of Construct 9 includes a stem duplex of 3 base pairs, wherein each nucleotide in the stem portion of the stem loop structure is a BNA, and a triloop. The structure of Construct 9 is identical to the structure of Construct 8, except that Construct 9 contains a triloop instead of a tetraloop.

FIG. 6E schematically shows the structure of an exemplary double-stranded nucleic acid inhibitor molecule (“Construct 10”) that targets a gene sequence of interest, as discussed in Example 2. The sense strand of Construct 10 includes a stem duplex of 2 base pairs, wherein each nucleotide in the stem portion of the stem loop structure is a BNA, and a tetraloop.

FIG. 6F schematically shows the structure of an exemplary double-stranded nucleic acid inhibitor molecule (“Construct 11”) that targets a gene sequence of interest, as discussed in Example 2. The sense strand of Construct 11 includes a stem duplex of 2 base pairs, wherein each nucleotide in the stem portion of the stem loop structure is a BNA, and a triloop. The structure of Construct 11 is identical to the structure of Construct 10, except that Construct 11 contains a triloop instead of a tetraloop.

FIG. 6G schematically shows the structure of an exemplary double-stranded nucleic acid inhibitor molecule (“Construct 12”) that targets a gene sequence of interest, as discussed in Example 2. The sense strand of Construct 12 includes a stem duplex of 1 base pair, wherein both nucleotides in the stem portion of the stem loop structure are a BNA, and a tetraloop.

FIG. 6H schematically shows the structure of an exemplary double-stranded nucleic acid inhibitor molecule (“Construct 13”) that targets a gene sequence of interest, as discussed in Example 2. The sense strand of Construct 13 includes a stem duplex of 1 base pair, wherein both nucleotides in the stem portion of the stem loop structure are a BNA, and a triloop. The structure of Construct 13 is identical to the structure of Construct 12, except that Construct 13 contains a triloop instead of a tetraloop.

FIG. 7 shows the percent of target gene mRNA remaining 4 days after administering Construct 1 (see FIG. 2A) and Constructs 6-13 (see FIGS. 6A-H) to CD-1 mice, as described in Example 2. The inclusion of a triloop in Constructs 7, 9, and 11 did not significantly reduce the potency of gene knockdown as compared to the tetraloop constructs 1, 6, 8, and 10, respectively and, in certain instances, actually improved potency. Construct 13, containing a triloop and a single base pair of bicyclic nucleotides in the stem duplex did not reduce target mRNA expression, in contrast to Construct 12, containing a tetraloop and a single base pair of bicyclic nucleotides in the stem duplex, which exhibited potent reduction of target mRNA expression.

FIG. 8 shows one non-limiting embodiment of a lipid nanoparticle (LNP) that can be used to formulate the double-stranded nucleic acid inhibitor molecule. The LNP includes the following core lipids: DL-048 (cationic lipid) and DSG-mPEG (pegylated lipid), and the following envelope lipids: DL-103 (cationic lipid), DSPC, cholesterol, and DSPE-mPEG (pegylated lipid).

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

Administer: As used herein, “administering” a composition to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Acyl: As used herein, the term “acyl” refers to an alkylcarbonyl, cycloalkylcarbonyl and arylcarbonyl moiety.

Alkoxy: As used herein, the term “alkoxy” refers to an alkyl group attached to a molecular moiety through an oxygen atom.

Alkenyl: As used herein, the term “alkenyl” refers to straight or branched chain hydrocarbyl groups having at least one carbon-carbon double bond, and having in the range of about 2 to about 20 carbon atoms. “Substituted alkenyl” refers to alkenyl groups further bearing one or more substituents. As used herein, “lower alkenyl” refers to alkenyl moieties having from 2 to about 6 carbon atoms.

Alkyl: As used herein, the term “alkyl” refers to straight or branched chain hydrocarbyl groups having from 1 up to about 20 carbon atoms. Whenever it appears herein, a numerical range, such as “C₁-C₆ alkyl” means that an alkyl group may comprise only 1 carbon atom, 2 carbon atoms, 3 carbon atoms, etc., up to and including 6 carbon atoms, although the term “alkyl” also includes instances where no numerical range of carbon atoms is designated. For example, the term “alkyl” can refer to a sub-range between C₁-C₁₀ (e.g. C₁-C₆). “Substituted alkyl” refers to alkyl moieties bearing substituents. As used herein, “lower alkyl” refers to alkyl moieties having from 1 to about 6 carbon atoms.

Alkynyl: As used herein, “alkynyl” refers to straight or branched chain hydrocarbyl groups having at least one carbon-carbon triple bond, and having in the range of about 2 to about 20 carbon atoms. “Substituted alkynyl” refers to alkynyl groups further bearing one or more substituents. As used herein, “lower alkynyl” refers to alkynyl moieties having from about 2 to about 6 carbon atoms.

Antisense strand: A double-stranded nucleic acid inhibitor molecule comprises two oligonucleotide strands: an antisense strand and a sense strand. The antisense strand or a region thereof is partially, substantially or fully complementary to a corresponding region of a target nucleic acid. In addition, the antisense strand of the double-stranded nucleic acid inhibitor molecule or a region thereof is partially, substantially or fully complementary to the sense strand of the double-stranded nucleic acid inhibitor molecule or a region thereof. In certain embodiments, the antisense strand may also contain nucleotides that are non-complementary to the target nucleic acid sequence. The non-complementary nucleotides may be on either side of the complementary sequence or may be on both sides of the complementary sequence. In certain embodiments, where the antisense strand or a region thereof is partially or substantially complementary to the sense strand or a region thereof, the non-complementary nucleotides may be located between one or more regions of complementarity (e.g., one or more mismatches). The antisense strand of a double-stranded nucleic acid inhibitor molecule is also referred to as the guide strand.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Aryl: As used herein, the term “aryl” refers to an aromatic monocyclic or multicyclic groups having in the range of 5 up to 19 carbon atoms. “Substituted aryl” refers to aryl groups further bearing one or more substituents.

Bicyclic nucleotide: As used herein, the term “bicyclic nucleotide” refers to a nucleotide comprising a bicyclic sugar moiety.

Bicyclic sugar moiety: As used herein, the term “bicyclic sugar moiety” refers to a modified sugar moiety comprising a 4 to 7 membered ring (including but not limited to a furanosyl) comprising a bridge connecting two atoms of the 4 to 7 membered ring to form a second ring, resulting in a bicyclic structure. Typically, the 4 to 7 membered ring is a sugar. In some embodiments, the 4 to 7 member ring is a furanosyl. In certain embodiments, the bridge connects the 2′-carbon and the 4′-carbon of the furanosyl.

Complementary: As used herein, the term “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. “Fully complementary” or 100% complementarity refers to the situation in which each nucleotide monomer of a first oligonucleotide strand or of a segment of a first oligonucleotide strand can form a base pair with each nucleotide monomer of a second oligonucleotide strand or of a segment of a second oligonucleotide strand. Less than 100% complementarity refers to the situation in which some, but not all, nucleotide monomers of two oligonucleotide strands (or two segments of two oligonucleotide strands) can form base pairs with each other. “Substantial complementarity” refers to two oligonucleotide strands (or segments of two oligonucleotide strands) exhibiting 90% or greater complementarity to each other. “Sufficiently complementary” refers to complementarity between a target mRNA and a nucleic acid inhibitor molecule, such that there is a reduction in the amount of protein encoded by a target mRNA.

Complementary strand: As used herein, the term “complementary strand” refers to a strand of a double-stranded nucleic acid inhibitor molecule that is partially, substantially or fully complementary to the other strand.

Cycloalkyl: As used herein, the term “cycloalkyl” refers to cyclic (i.e., ring-containing) hydrocarbon groups containing 3 to 12 carbons, for example, 3 to 8 carbons and, for example, 3 to 6 carbons. “Substituted cycloalkyl” refers to cycloalkyl groups further bearing one or more substituents.

Deoxyribofuranosyl: As used herein, the term “deoxyribofuranosyl” refers to a furanosyl that is found in naturally occurring DNA and has a hydrogen group at the 2′-carbon, as illustrated below:

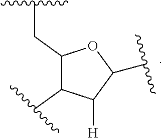 [SMILES: *CC1OC(*)CC1*]

Deoxyribonucleotide: As used herein, the term “deoxyribonucleotide” refers to a natural nucleotide (as defined herein) or modified nucleotide (as defined herein) which has a hydrogen group at the 2′-position of the sugar moiety.

dsRNAi inhibitor molecule: As used herein, the term “dsRNAi inhibitor molecule” refers to a double-stranded nucleic acid inhibitor molecule having a sense strand (passenger) and antisense strand (guide), where the antisense strand or part of the antisense strand is used by the Argonaute 2 (Ago2) endonuclease in the cleavage of a target mRNA.

Duplex: As used herein, the term “duplex,” in reference to nucleic acids (e.g., oligonucleotides), refers to a structure formed through complementary base pairing of two antiparallel sequences of nucleotides.

Excipient: As used herein, the term “excipient” refers to a non-therapeutic agent that may be included in a composition, for example to provide or contribute to a desired consistency or stabilizing effect.

Furanosyl: As used herein, the term “furanosyl” refers to a structure comprising a 5-membered ring with four carbon atoms and one oxygen atom.

Halo: As used herein, the terms “halo” and “halogen” are interchangeable and refer to an atom selected from fluorine, chlorine, bromine and iodine.

Heterocycle: As used herein, the terms “heterocycle” or “heterocyclic” refer to non-aromatic cyclic (i.e., ring-containing) groups containing one or more heteroatoms (e.g., N, O, S, or the like) as part of the ring structure, and having in the range of 3 up to 14 carbon atoms. “Substituted heterocyclic” or “substituted heterocycle” refer to heterocyclic groups further bearing one or more substituents.

Internucleotide linking group: As used herein, the term “internucleotide linking group” or “internucleotide linkage” refers to a chemical group capable of covalently linking two nucleoside moieties. Typically, the chemical group is a phosphorus-containing linkage group containing a phospho or phosphite group. Phospho linking groups are meant to include a phosphodiester linkage, a phosphorodithioate linkage, a phosphorothioate linkage, a phosphotriester linkage, a thionoalkylphosphonate linkage, a thionalkylphosphotriester linkage, a phosphoramidite linkage, a phosphonate linkage and/or a boranophosphate linkage. Many phosphorus-containing linkages are well known in the art, as disclosed, for example, in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050. In other embodiments, the oligonucleotide contains one or more internucleotide linking groups that do not contain a phosphorous atom, such short chain alkyl or cycloalkyl internucleotide linkages, mixed heteroatom and alkyl or cycloalkyl internucleotide linkages, or one or more short chain heteroatomic or heterocyclic internucleotide linkages, including, but not limited to, those having siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; and amide backbones. Non-phosphorous containing linkages are well known in the art, as disclosed, for example, in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

Loop: As used herein, the term “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length. Examples of loops include the unpaired nucleotides present in such structures as hairpins, tetraloops, and triloops.

Melting Temperature: As used herein, “melting temperature” or “Tₘ” means the temperature at which the two strands of a duplex nucleic acid separate. Tₘ is often used as a measure of duplex stability or the binding affinity of two strands of complementary nucleic acids or portions thereof. Tₘ can be measured by using the UV spectrum to determine the formation and breakdown (melting) of hybridization. Base stacking, which occurs during hybridization, is accompanied by a reduction in UV absorption (hypochromicity). Consequently, a reduction in UV absorption indicates a higher Tₘ.

Modified nucleobase: As used herein, the term “modified nucleobase” refers to any nucleobase that is not a natural nucleobase or a universal nucleobase. Suitable modified nucleobases include diaminopurine and its derivatives, alkylated purines or pyrimidines, acylated purines or pyrimidines thiolated purines or pyrimidines, and the like. Other suitable modified nucleobases include analogs of purines and pyrimidines. Suitable analogs include, but are not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, hypoxanthine, xanthine, 2-aminopurine, 6-hydroxyaminopurine, nitropyrrolyl, nitroindolyl and difluorotolyl, 6-thiopurine and 2,6-diaminopurine nitropyrrolyl, nitroindolyl and difluorotolyl. Typically a nucleobase contains a nitrogenous base. In certain embodiments, the nucleobase does not contain a nitrogen atom. See e.g., U.S. Published Patent Application No. 20080274462.

Modified nucleoside: As used herein, the term “modified nucleoside” refers to a heterocyclic nitrogenous base in N-glycosidic linkage with a sugar (e.g., deoxyribose or ribose or analog thereof) that is not linked to a phosphate group or a modified phosphate group (as defined herein) and that contains one or more of a modified nucleobase (as defined herein), a universal nucleobase (as defined herein) or a modified sugar moiety (as defined herein). The modified or universal nucleobases (also referred to herein as base analogs) are generally located at the 1 ‘-position of a nucleoside sugar moiety and refer to nucleobases other than adenine, guanine, cytosine, thymine and uracil at the 1’-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). Suitable modified or universal nucleobases or modified sugars in the context of the present disclosure are described herein.

Modified nucleotide: As used herein, the term “modified nucleotide” refers to a heterocyclic nitrogenous base in N-glycosidic linkage with a sugar (e.g., ribose or deoxyribose or analog thereof) that is linked to a phosphate group or a modified phosphate group (as defined herein) and contains one or more of a modified nucleobase (as defined herein), a universal nucleobase (as defined herein), or a modified sugar moiety (as defined herein). The modified or universal nucleobases (also referred to herein as base analogs) are generally located at the 1′-position of a nucleoside sugar moiety and refer to nucleobases other than adenine, guanine, cytosine, thymine and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). Suitable modified or universal nucleobases, modified sugar moieties, or modified phosphate groups in the context of the present disclosure are described herein.

Modified phosphate group: As used herein, the term “modified phosphate group” refers to a modification of the phosphate group that does not occur in natural nucleotides and includes non-naturally occurring phosphate mimics as described herein, including phosphate mimics that include a phosphorous atom and anionic phosphate mimics that do not include phosphate (e.g. acetate). Modified phosphate groups also include non-naturally occurring internucleotide linking groups, including both phosphorous-containing internucleotide linking groups, including, for example, phosphorothioate, and non-phosphorous containing linking groups, as described herein.

Modified sugar moiety: As used herein, a “modified sugar moiety” refers to a substituted sugar moiety (as defined herein) or a sugar analog (as defined herein).

Natural nucleobase: As used herein, the term “natural nucleobase” refers to the five primary, naturally occurring heterocyclic nucleobases of RNA and DNA, i.e., the purine bases: adenine (A) and guanine (G), and the pyrimidine bases: thymine (T), cytosine (C), and uracil (U).

Natural nucleoside: As used herein, the term “natural nucleoside” refers to a natural nucleobase (as defined herein) in N-glycosidic linkage with a natural sugar moiety (as defined herein) that is not linked to a phosphate group.

Natural nucleotide: As used herein, the term “natural nucleotide” refers to a natural nucleobase (as defined herein) in N-glycosidic linkage with a natural sugar moiety (as defined herein) that is linked to a phosphate group.

Natural sugar moiety: As used herein, the term “natural sugar moiety” refers to a ribofuranosyl (as defined herein) or a deoxyribofuranosyl (as defined herein).

Nucleic acid inhibitor molecule: As used herein, the term “nucleic acid inhibitor molecule” refers to an oligonucleotide molecule that reduces or eliminates the expression of a target gene wherein the oligonucleotide molecule contains a region that specifically targets a sequence in the target gene mRNA. Typically, the targeting region of the nucleic acid inhibitor molecule comprises a sequence that is sufficiently complementary to a sequence on the target gene mRNA to direct the effect of the nucleic acid inhibitor molecule to the specified target gene. The nucleic acid inhibitor molecule may include ribonucleotides, deoxyribonucleotides, and/or modified nucleotides.

Nucleobase: As used herein, the term “nucleobase” refers to a natural nucleobase (as defined herein), a modified nucleobase (as defined herein), or a universal nucleobase (as defined herein).

Nucleoside: As used herein, the term “nucleoside” refers to a natural nucleoside (as defined herein) or a modified nucleoside (as defined herein).

Nucleotide: As used herein, the term “nucleotide” refers to a natural nucleotide (as defined herein) or a modified nucleotide (as defined herein).

Overhang: As used herein, the term “overhang” refers to terminal non-base pairing nucleotide(s) at either end of either strand of a double-stranded nucleic acid inhibitor molecule. In certain embodiments, the overhang results from one strand or region extending beyond the terminus of the complementary strand to which the first strand or region forms a duplex. One or both of two oligonucleotide regions can form a duplex through hydrogen bonding of base pairs may have a 5′- and/or 3′-end that extends beyond the 3′- and/or 5′-end of complementarity shared by the two polynucleotides or regions. The single-stranded region extending beyond the 3′- and/or 5′-end of the duplex is referred to as an overhang.

Pharmaceutical composition: As used herein, the term “pharmaceutical composition” comprises a pharmacologically effective amount of a double-stranded nucleic acid inhibitor molecule and a pharmaceutically acceptable excipient (as defined herein).

Pharmaceutically acceptable excipient: As used herein, the term “pharmaceutically acceptable excipient” means that the excipient is one that is suitable for use with humans and/or animals without undue adverse side effects (such as toxicity, irritation, and allergic response) commensurate with a reasonable benefit/risk ratio.

Phosphate mimic: As used herein, the term “phosphate mimic” refers to a chemical moiety at the 5′-terminal end of an oligonucleotide that mimics the electrostatic and steric properties of a phosphate group. Many phosphate mimics have been developed that can be attached to the 5′-end of an oligonucleotide (see, e.g., U.S. Pat. No. 8,927,513; Prakash et al. Nucleic Acids Res., 2015,43(6):2993-3011). Typically, these 5′-phosphate mimics contain phosphatase-resistant linkages. Suitable phosphate mimics include 5′-phosphonates, such as 5′-methylenephosphonate (5′-MP) and 5′-(E)-vinylphosphonate (5′-VP) and 4′-phosphate analogs that are bound to the 4′-carbon of the sugar moiety (e.g., a ribose or deoxyribose or analog thereof) of the 5′-terminal nucleotide of an oligonucleotide, such as 4′-oxymethylphosphonate, 4′-thiomethylphosphonate, or 4′-aminomethylphosphonate, as described in International Publication No. WO 2018/045317, which is hereby incorporated by reference in its entirety. In certain embodiments, the 4′-oxymethylphosphonate is represented by the formula —O—CH₂—PO(OH)₂ or —O—CH₂—PO(OR)₂, where R is independently selected from H, CH₃, an alkyl group, or a protecting group. In certain embodiments, the alkyl group is CH₂CH₃. More typically, R is independently selected from H, CH₃, or CH₂CH₃. Other modifications have been developed for the 5′-end of oligonucleotides (see, e.g., WO 2011/133871).

Protecting group: As used herein, the term “protecting group” is used in the conventional chemical sense as a group which reversibly renders unreactive a functional group under certain conditions of a desired reaction. After the desired reaction, protecting groups may be removed to deprotect the protected functional group. All protecting groups should be removable under conditions which do not degrade a substantial proportion of the molecules being synthesized.

Reduce(s): The term “reduce” or “reduces” as used herein refers to its meaning as is generally accepted in the art. With reference to nucleic acid inhibitor molecules, the term generally refers to the reduction in the expression of a gene, or level of RNA molecules or equivalent RNA molecules encoding one or more proteins or protein subunits, or activity of one or more proteins or protein subunits, below that observed in the absence of the nucleic acid inhibitor molecules.

Ribofuranosyl: As used herein, the term “ribofuranosyl” refers to a furanosyl that is found in naturally occurring RNA and has a hydroxyl group at the 2′-carbon, as illustrated below:

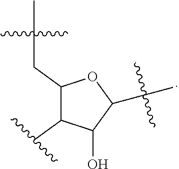

Ribonucleotide: As used herein, the term “ribonucleotide” refers to a natural nucleotide (as defined herein) or a modified nucleotide (as defined herein) which has a hydroxyl group at the 2′-position of the sugar moiety.

Sense strand: A double-stranded nucleic acid inhibitor molecule comprises two oligonucleotide strands: an antisense strand and a sense strand. The sense strand or a region thereof is partially, substantially or fully complementary to the antisense strand of the double-stranded nucleic acid inhibitor molecule or a region thereof. In certain embodiments, the sense strand may also contain nucleotides that are non-complementary to the antisense strand. The non-complementary nucleotides may be on either side of the complementary sequence or may be on both sides of the complementary sequence. In certain embodiments, where the sense strand or a region thereof is partially or substantially complementary to the antisense strand or a region thereof, the non-complementary nucleotides may be located between one or more regions of complementarity (e.g., one or more mismatches). The sense strand is also called the passenger strand.

Subject: As used herein, the term “subject” means any mammal, including mice, rabbits, and humans. In one embodiment, the subject is a human. The terms “individual” or “patient” are intended to be interchangeable with “subject.”

Substituent or substituted: The terms “substituent” or “substituted” as used herein refer to the replacement of hydrogen radicals in a given structure with the radical of a substituent. When more than one position in any given structure may be substituted with more than one substituent, the substituent may be either the same or different at every position unless otherwise indicated. As used herein, the term “substituted” is contemplated to include all permissible substituents that are compatible with organic compounds. The permissible substituents include acyclic and cyclic, branched and unbranched, carbocyclic and heterocyclic, aromatic and nonaromatic substituents of organic compounds. This disclosure is not intended to be limited in any manner by the permissible substituents of organic compounds.

Substituted sugar moiety: As used herein, a “substituted sugar moiety” includes furanosyls comprising one or more modifications. Typically, the modifications occur at the 2′-, 3′-, 4′-, or 5′-carbon position of the sugar. In certain embodiments, the substituted sugar moiety is a bicyclic sugar moiety comprising a bridge that connects the 2′-carbon with the 4-carbon of the furanosyl.

Sugar analog: As used herein, the term “sugar analog” refers to a structure that does not comprise a furanosyl and that is capable of replacing the naturally occurring sugar moiety of a nucleotide, such that the resulting nucleotide is capable of (1) incorporation into an oligonucleotide and (2) hybridization to a complementary nucleotide. Such structures typically include relatively simple changes to the furanosyl, such as rings comprising a different number of atoms (e.g., 4, 6, or 7-membered rings); replacement of the oxygen of the furanosyl with a non-oxygen atom (e.g., carbon, sulfur, or nitrogen); or both a change in the number of atoms and a replacement of the oxygen. Such structures may also comprise substitutions corresponding with those described for substituted sugar moieties. Sugar analogs also include more complex sugar replacements (e.g., the non-ring systems of peptide nucleic acid). Sugar analogs include without limitation morpholinos, cyclohexenyls and cyclohexitols.

Sugar moiety: As used herein, the term “sugar moiety” refers to a natural sugar moiety or a modified sugar moiety of a nucleotide or nucleoside.

Target site: As used herein, the term “target site” “target sequence,” “target nucleic acid”, “target region,” “target gene” are used interchangeably and refer to a RNA or DNA sequence that is “targeted,” e.g., for cleavage mediated by an RNAi inhibitor molecule that contains a sequence within its guide/antisense region that is partially, substantially, or perfectly or sufficiently complementary to that target sequence.

Tetraloop: As used herein, the term “tetraloop” refers to a loop (a single stranded region) that forms a stable secondary structure that contributes to the stability of an adjacent Watson-Crick hybridized nucleotides. Without being limited to theory, a tetraloop may stabilize an adjacent Watson-Crick base pair by stacking interactions. In addition, interactions among the nucleotides in a tetraloop include but are not limited to non-Watson-Crick base pairing, stacking interactions, hydrogen bonding, and contact interactions (Cheong et al., Nature 1990; 346(6285):680-2; Heus and Pardi, Science 1991; 253(5016):191-4). A tetraloop confers an increase in the melting temperature (Tm) of an adjacent duplex that is higher than expected from a simple model loop sequence consisting of random bases. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. A tetraloop may contain ribonucleotides, deoxyribonucleotides, modified nucleotides, and combinations thereof. In certain embodiments, a tetraloop consists of four nucleotides. In certain embodiments, a tetraloop consists of five nucleotides.

Examples of RNA tetraloops include the UNCG family of tetraloops (e.g., UUCG), the GNRA family of tetraloops (e.g., GAAA), and the CUYG family of tetraloops, including the CUUG tetraloop. (Woese et al., PNAS, 1990, 87(21):8467-71; Antao et al., Nucleic Acids Res., 1991, 19(21):5901-5). Other examples of RNA tetraloops include the GANC, A/UGNN, and UUUM tetraloop families (Thapar et al., WILEY INTERDISCIP. REV RNA, 2014, 5(1):1-28) and the GGUG, RNYA, and AGNN tetraloop families (Bottaro et al., BIOPHYS J., 2017, 113:257-67). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). (Nakano et al. Biochemistry, 2002, 41(48):14281-14292. Shinji et al., Nippon Kagakkai Koen Yokoshu, 2000, 78(2):731).

Tₘ-Increasing Nucleotide: As used herein, the term “Tₘ-increasing nucleotide” refers to a nucleotide that increases the melting temperature (Tₘ) of an oligonucleotide duplex as compared to the oligonucleotide duplex without the Tₘ-increasing nucleotide. Tₘ-increasing nucleotides include, but are not limited to, bicyclic nucleotides, tricyclic nucleotides, a G-clamp and analogues thereof, and hexitol nucleotides. Certain modified nucleotides having a modified sugar moiety or a modified nucleobase can also be used to increase the Tₘ of an oligonucleotide duplex. As used herein, the term “Tₘ-increasing nucleotide” specifically excludes nucleotides modified at the 2′-position of the sugar moiety with 2′-OMe or 2′-F.

Therapeutically effective amount: As used herein, a “therapeutically effective amount” or “pharmacologically effective amount” refers to that amount of a double-stranded nucleic acid inhibitor molecule effective to produce the intended pharmacological, therapeutic or preventive result.

Triloop: As used herein, the term “triloop” refers to a loop (a single stranded region) that forms a stable secondary structure that contributes to the stability of an adjacent Watson-Crick hybridized nucleotides and consists of three nucleotides. Without being limited to theory, a triloop may be stabilized by non-Watson-Crick base pairing of nucleotides within the triloop and base-stacking interactions. (Yoshizawa et al., Biochemistry 1997; 36, 4761-4767). A triloop can also confer an increase in the melting temperature (Tm) of an adjacent duplex that is higher than expected from a simple model loop sequence consisting of random bases. A triloop may contain ribonucleotides, deoxyribonucleotides, modified nucleotides, and combinations thereof. Examples of triloops include the GNA family of triloops (e.g., GAA, GTA, GCA, and GGA). (Yoshizawa 1997). In certain embodiments, the triloop has a nucleotide sequence of GAA.

Universal nucleobase: As used herein, a “universal nucleobase” refers to a base that can pair with more than one of the bases typically found in naturally occurring nucleic acids and can thus substitute for such naturally occurring bases in a duplex. The base need not be capable of pairing with each of the naturally occurring bases. For example, certain bases pair only or selectively with purines, or only or selectively with pyrimidines. The universal nucleobase may base pair by forming hydrogen bonds via Watson-Crick or non-Watson-Crick interactions (e.g., Hoogsteen interactions). Representative universal nucleobases include inosine and its derivatives.

### DETAILED DESCRIPTION

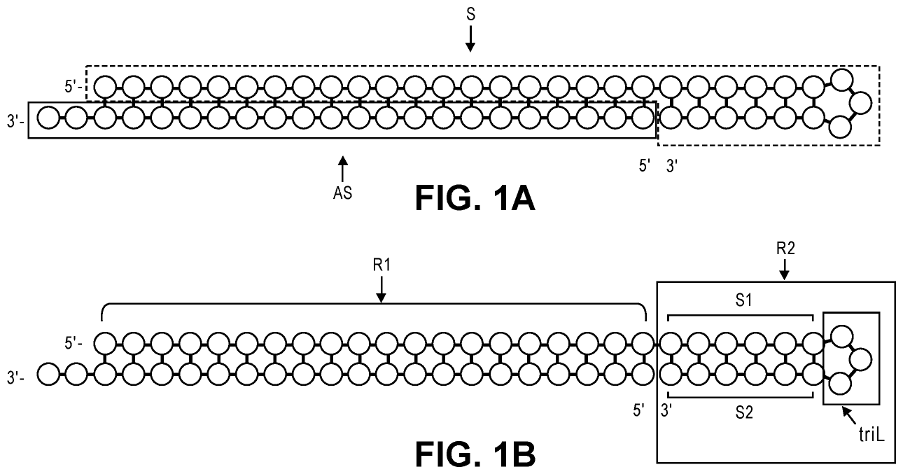

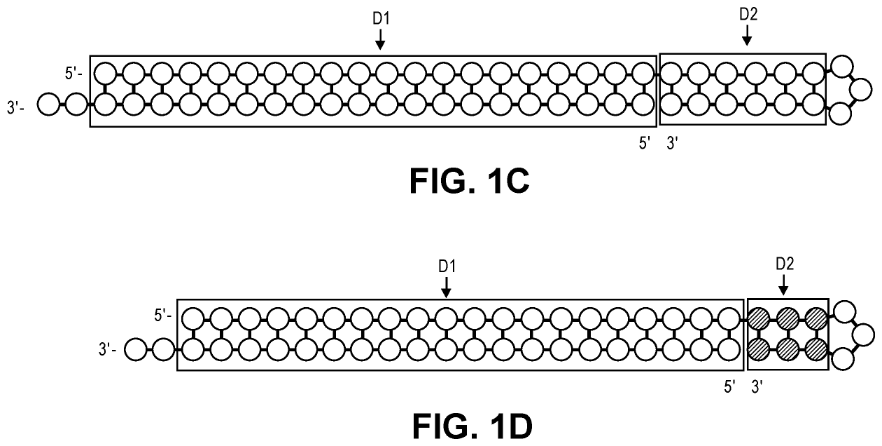

This application provides double-stranded nucleic acid inhibitor molecules having a sense strand with a stem loop structure and an antisense strand, where the loop portion of the stem loop structure is a triloop. The double-stranded nucleic acid inhibitor molecule contains a first duplex (D1) between a first region (R1) of the sense strand (S) and the antisense strand (AS) and a second duplex (D2) between a first subregion (S1) and a second subregion (S2) of a second region (R2) of the sense strand, where S1 and S2 are joined by the triloop (triL). See FIGS. 1A-D. Additionally, the stem portion of the stem loop structure may, in certain embodiments, contain at least one Tₘ-increasing nucleotide, such as from 4-12 Tₘ-increasing nucleotides, e.g. from 2-6 Tₘ-increasing nucleotide base pairs. The stem loop structure may be located at the 5′- or 3′-end of the sense strand. As disclosed herein, the double-stranded nucleic acid inhibitor molecules containing a triloop are active in reducing target gene expression. Moreover, in certain embodiments triloop-containing double-stranded nucleic acid inhibitor molecules may increase the potency of target gene expression as compared to their tetraloop-containing counterparts.

Also provided are methods of using the triloop containing double-stranded nucleic acid inhibitor molecules disclosed herein and compositions comprising the same to reduce the level or expression of a target gene in vitro or in vivo, including methods and compositions for treating diseases.

Nucleic Acid Inhibitor Molecules Containing a Triloop

This application discloses double-stranded nucleic acid inhibitor molecules having a sense strand with a stem loop structure and an antisense strand, wherein the loop portion of the stem loop structure is a triloop and wherein the sense strand and antisense strands are separate strands that each have a 5′- and 3′-end and, therefore, do not form a contiguous oligonucleotide. A typical stem/loop-containing double-stranded nucleic acid inhibitor molecule is shown in FIG. 1A with the sense strand (“S”) and antisense strand (“AS”) highlighted.

The sense strand can be further divided into a first region (R1) that forms a first duplex (D1) with the antisense strand (AS) and a second region (R2) that includes a loop (triL) that joins a first subregion (S1) with a second subregion (S2), as shown in FIGS. 1B and 1C. S1 and S2 are sufficiently complementary to each other to form a second duplex (D2), also referred to as the stem or stem duplex. See, e.g., FIGS. 1C and 1D. As described herein, the loop is a triloop. Typically, the triloop has the sequence GAA but other triloop sequences that confer an increase in the melting temperature (Tₘ) of an adjacent duplex that is higher than expected from a simple model loop sequence consisting of random bases may be used. The second duplex (D2) may contain at least one Tₘ-increasing nucleotide, and, in certain embodiments, all of the nucleotides in the second duplex (D2) may be Tₘ-increasing nucleotides. Typically, the double-stranded nucleic acid inhibitor molecule does not contain any Tₘ-increasing nucleotides outside of the second duplex (D2). In certain embodiments, the double-stranded nucleic acid molecule is a dsRNAi inhibitor molecule.

In certain embodiments of the double-stranded nucleic acid inhibitor molecule, the sense strand contains a stem duplex (D2) containing at least one Tₘ-increasing nucleotide and a triloop (triL) and is 20-65 nucleotides in length. In certain embodiments, the stem duplex is 2-6 base pairs in length. In certain embodiments, the antisense strand is 15-40 nucleotides in length.

In certain embodiments, the sense strand contains a stem duplex (D2) and triloop (triL) and is 20-65 nucleotides in length, and the antisense strand is 15-40 nucleotides in length. In certain embodiments, the extended part of the sense strand that contains the stem duplex (D2) and the triloop (triL) is on 3′-end of the strand. In certain other embodiments, the extended part of the sense strand that contains the stem (D2) and the triloop (triL) is on 5′-end of the strand.

In certain embodiments, the double-stranded nucleic acid inhibitor molecule comprises a sense strand and an antisense strand, wherein the sense and antisense strands are separate strands and form a first duplex (D1) of 18-24 base pairs, wherein the sense strand comprises a second duplex (D2) and a triloop (triL) and is 27-35 nucleotides in length, and wherein the antisense strand is 20-24 nucleotides in length. In certain embodiments, the sense strand is 27-35 nucleotides in length. In certain embodiments, the sense strand is 27-33 nucleotides in length. In certain embodiments, the sense strand is 29-31 nucleotides in length. In certain embodiments, the second duplex (D2) has a length of 2-6 base pairs. In certain embodiments, the second duplex (D2) has a length of 2 base pairs and contains Tₘ-increasing nucleotides. In certain embodiments, the second duplex (D2) has a length of 3 base pairs and contains Tₘ-increasing nucleotides. In certain embodiments, the second duplex (D2) has a length of 4 base pairs and contains Tₘ-increasing nucleotides. In certain embodiments, the second duplex (D2) has a length of 5 base pairs and contains Tₘ-increasing nucleotides. In certain embodiments, the second duplex (D2) has a length of 6 base pairs and does not contain any Tₘ-increasing nucleotides. In certain embodiments, the antisense strand has a single-stranded overhang of 1, 2, 3, or 4 nucleotides at its 3′-end. Typically, the single-stranded overhang at the 3′-end of the antisense strand consists of 2 nucleotides.

In certain embodiments, the double-stranded nucleic acid inhibitor molecule comprises a sense strand and an antisense strand, wherein the sense and antisense strands are separate strands and form a first duplex (D1) of 18-22 base pairs, such as 20 nucleotides, wherein the sense strand comprises a second duplex (D2) and a triloop (triL) and is 27-35 nucleotides in length, and wherein the antisense strand is 20-24 nucleotides in length, such as 22 nucleotides in length. In certain embodiments, D1 has a length of 19-21 base pairs. In certain embodiments, the antisense strand is 20-22 nucleotides in length. In certain embodiments, the sense strand is 29-33 nucleotides in length. In certain embodiments, the second duplex (D2) has a length of 2-6 base pairs. In certain embodiments, D2 has a length of 2 base pairs and contains Tₘ-increasing nucleotides. In certain embodiments, D2 has a length of 3 base pairs and contains Tₘ-increasing nucleotides. In certain embodiments, D2 has a length of 4 base pairs and contains Tₘ-increasing nucleotides. In certain embodiments, D2 has a length of 5 base pairs and contains Tₘ-increasing nucleotides. In certain embodiments, D2 has a length of 6 base pairs and does not contain any Tₘ-increasing nucleotides. In certain embodiments, the antisense strand has a single-stranded overhang of 1-5 nucleotides at its 3′-end. In certain embodiments, the antisense strand has a single-stranded overhang of 1, 2, 3, or 4 nucleotides at its 3′-end. Typically, the single-stranded overhang at the 3′-end of the antisense strand consists of 2 nucleotides.

In certain embodiments, the double-stranded nucleic acid inhibitor molecule comprises a sense strand and an antisense strand, wherein the sense and antisense strands are separate strands and form a first duplex (D1) of 19-21 base pairs, wherein the sense strand has a first region (R1) of 19-21 nucleotides and a second region (R2) of 7-15 nucleotides that comprises a triloop (triL) that joins a first subregion (S1) to a second subregion (S2), wherein each of S1 and S2 is 2-6 nucleotides in length and are sufficiently complementary to each other to form a second duplex (D2), and wherein the antisense strand is 20-24 nucleotides in length. In certain embodiments, the antisense strand has a single-stranded overhang of two nucleotides at its 3′-end. In certain embodiments, each of S1 and S2 is 2-5 nucleotides in length. In certain embodiments, D2 has a length of 2 base pairs and contains Tₘ-increasing nucleotides. In certain embodiments, D2 has a length of 3 base pairs and contains Tₘ-increasing nucleotides. In certain embodiments, D2 has a length of 4 base pairs and contains Tₘ-increasing nucleotides. In certain embodiments, D2 has a length of 5 base pairs and contains Tₘ-increasing nucleotides. In certain embodiments, D2 has a length of 6 base pairs and does not contain Tₘ-increasing nucleotides.

In certain embodiments, the double-stranded nucleic acid inhibitor molecule comprises a sense strand and an antisense strand, wherein the sense and antisense strands are separate strands and form a first duplex (D1) of 20 base pairs, wherein the sense strand has a first region (R1) of 20 nucleotides and a second region (R2) of 7-9 nucleotides that comprises a triloop (triL) that joins a first subregion (S1) to a second subregion (S2), wherein the antisense strand is 22 nucleotides in length and has a single-stranded overhang of two nucleotides at its 3′-end and wherein each of S1 and S2 contain bicyclic nucleotides. In certain embodiments, each of S1 and S2 is 3 nucleotides in length and form a second duplex (D2) of three base pairs. In certain embodiments, each of S1 and S2 is 2 nucleotides in length and form a second duplex (D2) of two base pairs. In certain embodiments, each nucleotide in the second duplex (D2) is a Tₘ-increasing nucleotide and the double-stranded nucleic acid inhibitor molecule does not contain any Tₘ-increasing nucleotides outside of the second duplex (D2).

In certain embodiments of the triloop-containing double-stranded nucleic acid inhibitor molecule described herein, the second duplex (D2) has a length of 2-6 base pairs. In certain embodiments, D2 has a length of 2-4 base pairs. In certain embodiments, D2 has a length of 2 base pairs. In certain embodiments, D2 has a length of 3 base pairs. In certain embodiments, D2 has a length of 4 base pairs. In certain embodiments, D2 has a length of 5 base pairs. In certain embodiments, D2 has a length of 6 base pairs.

In certain embodiments of the triloop-containing double-stranded nucleic acid inhibitor molecule described herein, the second duplex (D2) contains 4-10 Tₘ-increasing nucleotides and has a length of 2-5 base pairs. In certain embodiments, D2 contains 6-8 Tₘ-increasing nucleotides and has a length of 3-4 base pairs. In certain embodiments, D2 contains 6 Tₘ-increasing nucleotides and has a length of 3 base pairs. In certain embodiments, D2 contains 4 Tₘ-increasing nucleotides and has a length of 2 base pairs. In certain embodiments, each nucleotide in D2 is a Tₘ-increasing nucleotide.

In certain embodiments of the triloop-containing double-stranded nucleic acid inhibitor molecule described herein, the second duplex (D2) contains a single Tₘ-increasing nucleotide and has a length of 2-6 base pairs. In certain embodiments of the triloop-containing double-stranded nucleic acid inhibitor molecule described herein, the second duplex (D2) contains 2-6 single Tₘ-increasing nucleotide and has a length of 2-6 base pairs, wherein none of the Tₘ-increasing nucleotides in D2 form a base pair. For example, D2 may contain 2 Tₘ-increasing nucleotides and have a length of 2-6 base pairs, wherein the 2 Tₘ-increasing nucleotides do not form a base pair. D2 may also contain 3 Tₘ-increasing nucleotides and have a length of 3-6 base pairs, wherein the 3 Tₘ-increasing nucleotides do not form a base pair. D2 may also contain 4 Tₘ-increasing nucleotides and have a length of 4-6 base pairs, wherein the 4 Tₘ-increasing nucleotides do not form a base pair. D2 may also contain 5 Tₘ-increasing nucleotides and have a length of 5-6 base pairs, wherein the 5 Tₘ-increasing nucleotides do not form a base pair. D2 may also contain 6 Tₘ-increasing nucleotides and have a length of 6 base pairs, wherein the 6 Tₘ-increasing nucleotides do not form a base pair.

In certain embodiments, the triloop-containing double-stranded nucleic acid inhibitor molecule does not contain any Tₘ-increasing nucleotides. In certain embodiments, the double-stranded nucleic acid inhibitor molecule does not contain any Tₘ-increasing nucleotides in the first region of the sense strand (R1) or the antisense strand. In certain embodiments, the double-stranded nucleic acid inhibitor molecule does not contain any Tₘ-increasing nucleotides outside of the second duplex (D2). In certain embodiments, the double-stranded nucleic acid inhibitor molecule does not contain any Tₘ-increasing nucleotides in the second duplex (D2).

The one or more Tₘ-increasing nucleotides in the second duplex (D2) of the triloop-containing double-stranded nucleic acid molecule can be any of the Tₘ-increasing nucleotides described herein or otherwise available in the art. In certain embodiments, the double-stranded nucleic acid molecule contains at least two Tₘ-increasing nucleotides in the second duplex (D2) and each Tₘ-increasing nucleotide in the second duplex is the same. In certain embodiments, the double-stranded nucleic acid molecule contains at least two different Tₘ-increasing nucleotides in the second duplex (D2).

In any of the triloop-containing double-stranded nucleic acid molecules described herein, the one or more Tₘ-increasing nucleotides can be any of the bicyclic nucleotides described herein or otherwise available in the art. In any of the triloop-containing double-stranded nucleic acid moleculse described herein, the at least one bicyclic nucleotide in the second duplex (D2) comprises a bicyclic sugar moiety, wherein the bicyclic sugar moiety is a substituted furanosyl comprising a bridge that connects the 2′-carbon and the 4′-carbon of the furanosyl.

In any of the triloop-containing double-stranded nucleic acid inhibitor molecules described herein, the at least one bicyclic nucleotide in the second duplex (D2) has the structure of Formula I, II, III, IV, Va, or Vb. For example, the at least one bicyclic nucleotide in the second duplex (D2) can have the structure of Formula I. The at least one bicyclic nucleotide in the second duplex (D2) can also have the structure of Formula II. The at least one bicyclic nucleotide in the second duplex (D2) can also have the structure of Formula III. The at least one bicyclic nucleotide in the second duplex (D2) can also have the structure of Formula IV. The at least one bicyclic nucleotide in the second duplex (D2) can also have the structure of Formula Va. The at least one bicyclic nucleotide in the second duplex (D2) can also have the structure of Formula Vb.

The at least one bicyclic nucleotide in the second duplex (D2) can also the structure of one or more of Formula Ia, Ib, Ic, Id, Ie, or If. The at least one bicyclic nucleotide in the second duplex (D2) can also have the structure of one or more of Formula IIa, IIb, IIc, or IId. The at least one bicyclic nucleotide in the second duplex (D2) can also have the structure of Formula IIIa and/or IIIb. The at least one bicyclic nucleotide in the second duplex (D2) can also have the structure of Formula IVa and/or IVb.

In any of the triloop-containing double-stranded nucleic acid molecules described herein, the at least one bicyclic nucleotide (BN) in the second duplex (D2) is one or more of the following: (a) methyleneoxy BN, (b) ethyleneoxy BN, (c) aminooxy BN; (d) oxyamino BN, (e) methyl(methyleneoxy) BN (also known as constrained ethyl or cET), (f) methylene-thio BN, (g) methylene amino BN, (h) methyl carbocyclic BN, and (i) propylene carbocyclic BN. In one embodiment, the at least one BN is (a) methyleneoxy BN or (d) oxyamino BN), wherein R₂ is CH₃. For example, the at least one BN in D2 is the oxyamino BN (d), wherein R₂ is CH₃.

Bicyclic Nucleotides

The triloop-containing double-stranded nucleic acid inhibitor molecules disclosed herein contain a sense strand and an antisense strand and, in certain embodiments, may contain at least one bicyclic nucleotide in the stem portion of a stem loop structure that is present in the sense strand. Bicyclic nucleotides typically have a sugar moiety with a 4 to 7 membered ring (including but not limited to furanosyl) comprising a bridge connecting two atoms of the 4 to 7 membered ring to form a second ring, resulting in a bicyclic structure. In certain embodiments, the bridge connects the 2′-carbon and the 4′-carbon of the first ring to form a second ring. Such bicyclic nucleotides have various names including BNA's and LNA's for bicyclic nucleic acids and locked nucleic acids, respectively. The synthesis of bicyclic nucleotides and their incorporation into nucleic acid compounds has also been reported in the literature, including, for example, Singh et al., CHEM. COMMUN., 1998, 4, 455-456; Koshkin et al., TETRAHEDRON, 1998, 54, 3607-3630; Wahlestedt et al., PROC. NATL. ACAD. SCI. U.S.A., 2000, 97, 5633-5638; Kumar et al., BIOORG. MED. CHEM. LETT., 1998, 8, 2219-2222; Singh et al., J. ORG. CHEM., 1998, 63, 10035-10039; U.S. Pat. Nos. 7,427,672, 7,053,207, 6,794,499, 6,770,748, 6,268,490 and 6,794,499; and published U.S. applications 20040219565, 20040014959, 20030207841, 20040192918, 20030224377, 20040143114 and 20030082807; each of which is incorporated by reference herein, in its entirety.

Typically, the bridge contains 2 to 8 atoms. In certain embodiments, the bridge contains 3 atoms. In certain embodiments, the bridge contains 4 atoms. In certain embodiments, the bridge contains 5 atoms. In certain embodiments, the bridge contains 6 atoms. In certain embodiments, the bridge contains 7 atoms. In certain embodiments, the bridge contains 8 atoms. In certain embodiments, the bridge contains more than 8 atoms.

In certain embodiments, the bicyclic sugar moiety is a substituted furanosyl comprising a bridge that connects the 2′-carbon and the 4′-carbon of the furanosyl to form the second ring. In certain embodiments, the bicyclic nucleotide has the structure of Formula I:

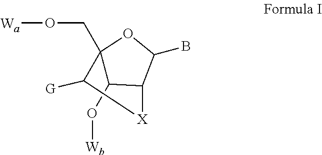

wherein B is a nucleobase;

wherein G is H, OH, NH₂, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol, or substituted thio;

wherein X is O, S, or NR₁, wherein R₁ is H, C₁-C₆ alkyl, C₁-C₆ alkoxy, benzene or pyrene; and

wherein Wₐ and W_{b} are each independently, H, OH, a hydroxyl protecting group, a phosphorous moiety, or an internucleotide linking group attaching the nucleotide represented by Formula I to another nucleotide or to an oligonucleotide and wherein at least one of Wₐ or W_{b} is an internucleotide linking group attaching the nucleotide represented by Formula I to an oligonucleotide.

In certain embodiments of Formula I, G is H and X is NR₁, wherein R₁ is benzene or pyrene. In certain embodiments, of Formula I, G is H and X is S.

In certain embodiments of Formula I, G is H and X is O:

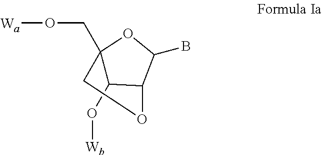

In certain embodiments of Formula I, G is H and X is NR₁, wherein R₁ is H, CH₃, or OCH₃:

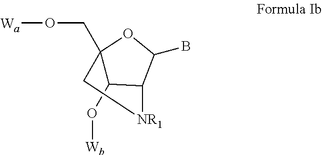

In certain embodiments of Formula I, G is OH or NH₂ and X is O.

In certain embodiments of Formula I, G is OH and X is O:

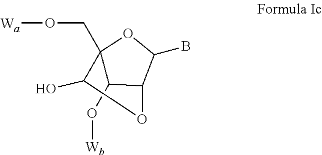

In certain embodiments of Formula I, G is NH₂ and X is O:

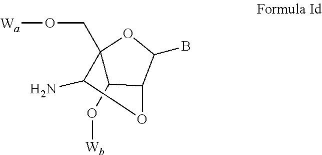

In certain embodiments, of Formula I, G is CH₃ or CH₂OCH₃ and X is O. In certain embodiments, of Formula I, G is CH₃ and X is O:

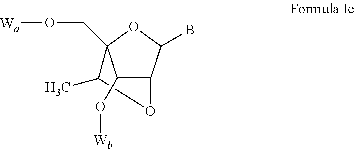

In certain embodiments, of Formula I, G is CH₂OCH₃ and X is O:

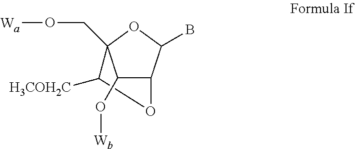

In certain embodiments, the bicyclic nucleotide has the structure of Formula II:

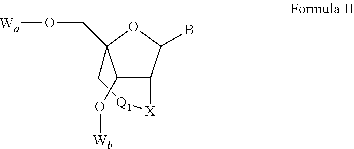

wherein B is a nucleobase;

wherein Q₁ is CH₂ or O;

wherein X is CH₂, O, S, or NR₁, wherein R₁ is H, C₁-C₆ alkyl, C₁-C₆ alkoxy, benzene or pyrene;

wherein if Q₁ is O, X is CH₂;

wherein if Q₁ is CH₂, X is CH₂, O, S, or NR₁, wherein R₁ is H, C₁-C₆ alkyl, C₁-C₆ alkoxy, benzene or pyrene;

wherein Wₐ and W_{b} are each independently, H, OH, a hydroxyl protecting group, a phosphorous moiety, or an internucleotide linking group attaching the nucleotide represented by Formula II to another nucleotide or to an oligonucleotide and wherein at least one of Wₐ or W_{b} is an internucleotide linking group attaching the nucleotide represented by Formula II to an oligonucleotide.

In certain embodiments of Formula II, Q₁ is O and X is CH₂:

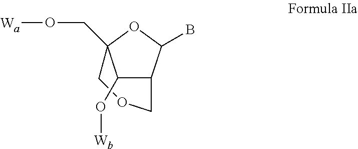

In certain embodiments of Formula II, Q₁ is CH₂ and X is O:

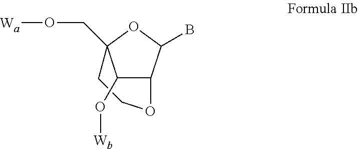

In certain embodiments of Formula II, Q₁ is CH₂ and X is NR₁, wherein R₁ is H, CH₃ or OCH₃:

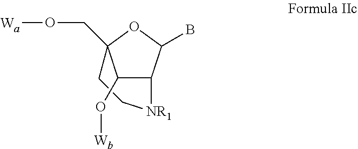

In certain embodiments of Formula II, Q₁ is CH₂ and X is NH:

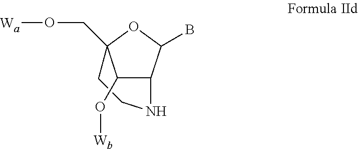

In certain embodiments, the bicyclic nucleotide has the structure of Formula

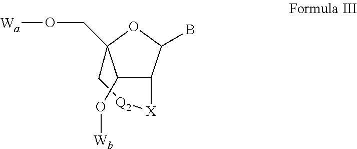

wherein B is a nucleobase;

wherein Q₂ is O or NR₁, wherein R₁ is H, C₁-C₆ alkyl, C₁-C₆ alkoxy, benzene or pyrene;

wherein X is CH₂, O, S, or NR₁, wherein R₁ is H, C₁-C₆ alkyl, C₁-C₆ alkoxy, benzene or pyrene;

wherein if Q₂ is O, X is NR₁;

wherein if Q₂ is NR₁, X is O or S;

wherein Wₐ and W_{b} are each independently, H, OH, a hydroxyl protecting group, a phosphorous moiety, or an internucleotide linking group attaching the nucleotide represented by Formula III to another nucleotide or to an oligonucleotide and wherein at least one of Wₐ or W_{b} is an internucleotide linking group attaching the nucleotide represented by Formula III to an oligonucleotide.

In certain embodiments of Formula III, Q₂ is O and X is NR₁. In certain embodiments of Formula III, Q₂ is O and X is NR₁, wherein R₁ is C₁-C₆ alkyl. In certain embodiments of Formula III, Q₂ is O and X is NR₁ and R₁ is H or CH₃.

In certain embodiments of Formula III, Q₂ is O and X is NR₁ and R₁ is CH₃:

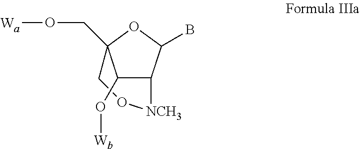

In certain embodiments of Formula III, Q₂ is NR₁ and X is O. In certain embodiments of Formula III, Q₂ is NR₁, wherein R₁ is C₁-C₆ alkyl and X is O.

In certain embodiments of Formula III, Q₂ is NCH₃ and X is O:

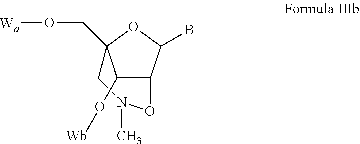

In certain embodiments, the bicyclic nucleotide has the structure of Formula IV:

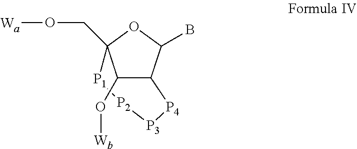

wherein B is a nucleobase;

wherein P₁ and P₃ are CH₂, P₂ is CH₂ or O and P₄ is O; and

wherein Wₐ and W_{b} are each independently, H, OH, a hydroxyl protecting group, a phosphorous moiety, or an internucleotide linking group attaching the nucleotide represented by Formula IV to another nucleotide or to an oligonucleotide and wherein at least one of Wₐ or W_{b} is an internucleotide linking group attaching the nucleotide represented by Formula IV to an oligonucleotide.

In certain embodiments of Formula IV, P₁, P₂, and P₃ are CH₂, and P₄ is O:

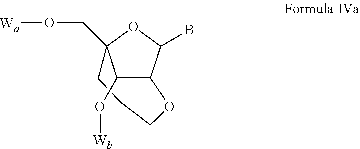

In certain embodiments of Formula IV, P₁ and P₃ are CH₂, P₂ is O and P₄ is O:

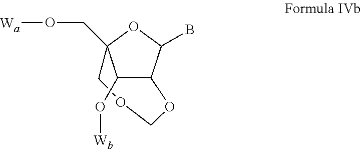

In certain embodiments, the bicyclic nucleotide has the structure of Formula Va or Vb:

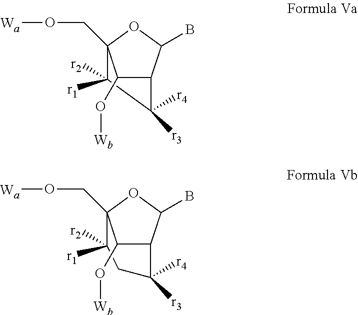

wherein B is a nucleobase;

wherein r1, r2, r3, and r4 are each independently H, halogen, C₁-C₁₂ alkyl, substituted C₁-C₁₂ alkyl, C₂-C₁₂ alkenyl, substituted C₂-C₁₂ alkenyl, C₂-C₁₂ alkynyl; substituted C₂-C₁₂ alkynyl; C₁-C₁₂ alkoxy; substituted C₁-C₁₂ alkoxy, OT₁, ST₁, SOT₁, SO₂T₁, NT₁T₂, N3, CN, C(═O)OT₁, C(═O)NT₁T₂, C(═O)T₁, O—C(═O)NT₁T₂, N(H)C(═NH)NT₁T₂, N(H)C(═O)NT₁T₂ or N(H)C(═S)NT₁T₂, wherein each of T₁ and T₂ is independently H, C₁-C₆ alkyl, or substituted C₁-C₁₆ alkyl; or

r1 and r2 or r3 and r4 together are ═C(r5)(r6), wherein r5 and r6 are each independently H, halogen, C₁-C₁₂ alkyl, or substituted C₁-C₁₂ alkyl; and

wherein Wₐ and W_{b} are each independently, H, OH, a hydroxyl protecting group, a phosphorous moiety, or an internucleotide linking group attaching the nucleotide represented by Formula V to another nucleotide or to an oligonucleotide and wherein at least one of Wₐ or W_{b} is an internucleotide linking group attaching the nucleotide represented by Formula V to an oligonucleotide.

In certain embodiments, the bicyclic sugar moiety is a substituted furanosyl comprising a bridge that connects the 2′-carbon and the 4′-carbon of the furanosyl to form the second ring, wherein the bridge that connects the 2′-carbon and the 4′-carbon of the furanosyl includes, but is not limited to:
a) 4′-CH₂—O—N(R)-2′ and 4′-CH₂—N(R)—O-2′, wherein R is H, C₁-C₁₂ alkyl, or a protecting group, including, for example, 4′-CH₂—NH—O-2′ (also known as BNA^{NC}), 4′-CH₂—N(CH₃)—O-2′ (also known as BNA^{NC}[NMe]), (as described in U.S. Pat. No. 7,427,672, which is hereby incorporated by reference in its entirety);
b) 4′-CH₂-2; 4′—(CH₂)₂-2; 4′—(CH₂)₃-2; 4′—(CH₂)—O-2′ (also known as LNA); 4′—(CH₂)—S-2; 4′—(CH₂)₂—O-2′ (also known as ENA); 4′-CH(CH₃)—O-2′ (also known as cEt); and 4′-CH(CH₂OCH₃)—O-2′ (also known as cMOE), and analogs thereof (as described in U.S. Pat. No. 7,399,845, which is hereby incorporated by reference in its entirety);
c) 4′-C(CH₃)(CH₃)—O-2′ and analogs thereof (as described in U.S. Pat. No. 8,278,283, which is hereby incorporated by reference in its entirety);
d) 4′-CH₂—N(OCH₃)-2′ and analogs thereof (as described in U.S. Pat. No. 8,278,425, which is hereby incorporated by reference in its entirety);
e) 4′-CH₂—O—N(CH₃)-2′ and analogs thereof (as described in U.S. Patent Publication No. 2004/0171570, which is hereby incorporated by reference in its entirety);
f) 4′-CH₂—C(H)(CH₃)-2′ and analogs thereof (as described in Chattopadhyaya et al., J. Org. Chem., 2009, 74, 118-34, which is hereby incorporated by reference in its entirety); and
g) 4′-CH₂—C(═CH₂)-2′ and analogs thereof as described in U.S. Pat. No. 8,278,426, which is hereby incorporated by reference in its entirety).

In certain embodiments, the bicyclic nucleotide (BN) is one or more of the following: (a) methyleneoxy BN, (b) ethyleneoxy BN, (c) aminooxy BN; (d) oxyamino BN, (e) methyl(methyleneoxy) BN (also known as constrained ethyl or cET), (0 methylene-thio BN, (g) methylene amino BN, (h) methyl carbocyclic BN, and (i) propylene carbocyclic BN, as shown below.

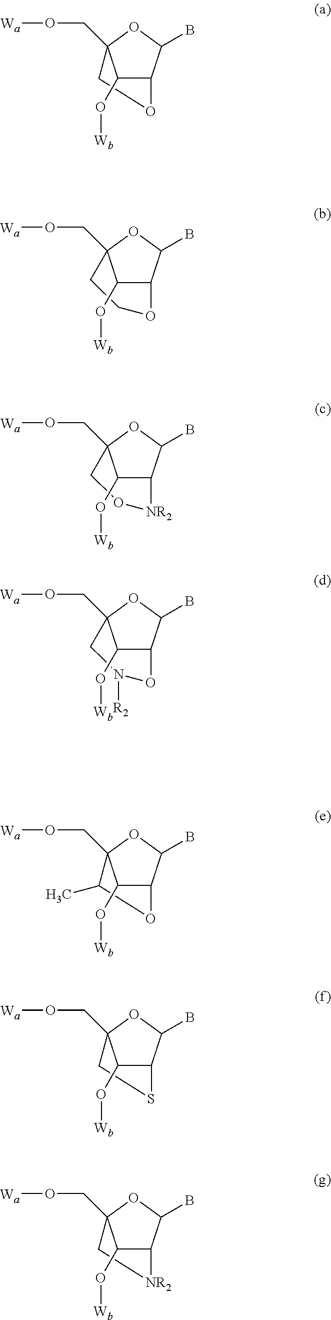

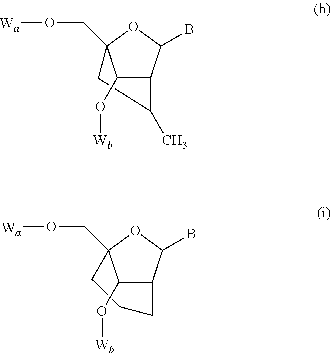

In the bicyclic nucleotides of (a) to (i) above, B is a nucleobase, R₂ is H or CH₃ and Wₐ and W_{b} are each independently, H, OH, a hydroxyl protecting group, a phosphorous moiety, or an internucleotide linking group attaching the bicyclic nucleotide to another nucleotide or to an oligonucleotide and wherein at least one of Wₐ or W_{b} is an internucleotide linking group attaching the bicyclic nucleotide to an oligonucleotide.

In one embodiment of the oxyamino BN (d), R₂ is CH₃, as follows (also known as BNA^{NC}[NMe]):

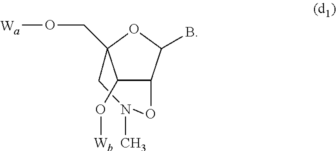

In certain embodiments, bicyclic sugar moieties and bicyclic nucleotides incorporating such bicyclic sugar moieties are further defined by isomeric configuration. In certain embodiments, the bicyclic sugar moiety or nucleotide is in the α-L configuration. In certain embodiments, the bicyclic sugar moiety or nucleotide is in the β-D configuration. For example, in certain embodiments, the bicyclic sugar moiety or nucleotide comprises a 2′O,4′-C-methylene bridge (2′-O—CH₂-4′) in the α-L configuration (α-L LNA). In certain embodiments, the bicyclic sugar moiety or nucleotide is in the R configuration. In certain embodiments, the bicyclic sugar moiety or nucleotide is in the S configuration. For example, in certain embodiments, the bicyclic sugar moiety or nucleotide comprises a 4′-CH(CH₃)—O-2′ bridge (i.e., cEt) in the S-configuration.

Tricyclic Nucleotides

In certain embodiments, the Tₘ-increasing nucleotide can be a tricyclic nucleotide. The synthesis of tricyclic nucleotides and their incorporation into nucleic acid compounds has also been reported in the literature, including, for example, Steffens et al., J. AM. CHEM. SOC. 1997; 119:11548-11549; Steffens et al., J. ORG. CHEM. 1999; 121(14):3249-3255; Renneberg et al., J. Am. CHEM. SOC. 2002; 124:5993-6002; Ittig et al., NUCLEIC ACIDS RES. 2004; 32(1):346-353; Scheidegger et al., Chemistry 2006; 12:8014-8023; Ivanova et al., OLIGONUCLEOTIDES 2007; 17:54-65; each of which is each hereby incorporated by reference in its entirety.

In certain embodiments, the tricyclic nucleotide is a tricyclo nucleotide (also called tricyclo DNA) in which the 3′-carbon and 5′-carbon centers are connected by an ethylene that is fused to a cyclopropane ring, as discussed for example in Leumann C J, Bioorg. Med. Chem. 2002; 10:841-854 and published U.S. Applications 2015/0259681 and 2018/0162897, which are each hereby incorporated by reference. In certain embodiments, the tricyclic nucleotide comprises a substituted furanosyl ring comprising a bridge that connects the 2′-carbon and the 4′-carbon of the furanosyl to form a second ring, and a third fused ring resulting from a group connecting the 5′-carbon to the methylene group of the bridge that connects the 2′-carbon and the 4′-carbon of the furanosyl, as discussed, for example, in published U.S. Application 2015/0112055, which is hereby incorporated by reference.

Other Tₘ-Increasing Nucleotides

In addition to bicyclic and tricyclic nucleotides, other Tₘ-increasing nucleotides can be used in the nucleic acid inhibitor molecules described herein. For example, in certain embodiments, the Tₘ-increasing nucleotide is a G-clamp, guanidine G-clamp or analogue thereof (Wilds et al., Chem, 2002; 114:123 and Wilds et al., Chim Acta 2003; 114:123), a hexitol nucleotide (Herdewijn, Chem. Biodiversity 2010; 7:1-59), or a modified nucleotide. The modified nucleotide can have a modified nucleobase, as described herein, including for example, 5-bromo-uracil, 5-iodo-uracil, 5-propynyl-modified pyrimidines, or 2-amino adenine (also called 2,6-diaminopurine) (Deleavey et al., Chem. & Biol. 2012; 19:937-54) or 2-thio uridine, 5 Me-thio uridine, and pseudo uridine. The modified nucleotide can also have a modified sugar moiety, as described for example, in U.S. Pat. No. 8,975,389, which is hereby incorporated by reference, or as described herein, except that the Tₘ-increasing nucleotide is not modified at the 2′-carbon of the sugar moiety with a 2′-F or a 2′-OMe.

In certain embodiments, the Tₘ-increasing nucleotide is a bicyclic nucleotide. In certain embodiments, the Tₘ-increasing nucleotide is a tricyclic nucleotide. In certain embodiments, the Tₘ-increasing nucleotide a G-clamp, guanidine G-clamp or analogue thereof. In certain embodiments, the Tₘ-increasing nucleotide is a hexitol nucleotide. In certain embodiments, the Tₘ-increasing nucleotide is a bicyclic or tricyclic nucleotide. In certain embodiments, the Tₘ-increasing nucleotide is a bicyclic nucleotide, a tricyclic nucleotide, or a G-clamp, guanidine G-clamp or analogue thereof. In certain embodiments, the Tₘ-increasing nucleotide is a bicyclic nucleotide, a tricyclic nucleotide, a G-clamp, guanidine G-clamp or analogue thereof, or a hexitol nucleotide.

In certain embodiments, the Tₘ-increasing nucleotide increases the Tₘ of the second duplex (D2) of the nucleic acid inhibitor molecule by at least 2° C. per incorporation. In certain embodiments, the Tₘ-increasing nucleotide increases the Tₘ of D2 by at least 3° C. per incorporation. In certain embodiments, the Tₘ-increasing nucleotide increases the Tₘ of D2 by at least 4° C. per incorporation. In certain embodiments, the Tₘ-increasing nucleotide increases the Tₘ of D2 by at least 5° C. per incorporation.

Other Modifications

The double-stranded nucleic acid inhibitor molecules described herein can contain other nucleotide modifications in addition to the at least one Tₘ-increasing nucleotide in the second duplex (D2). Typically, multiple nucleotides of the double-stranded nucleic acid inhibitor molecule are modified to improve various characteristics of the molecule such as resistance to nucleases or lowered immunogenicity. See, e.g., Bramsen et al. (2009), Nucleic Acids Res., 37, 2867-2881. Many nucleotide modifications have been used in the oligonucleotide field, particularly for nucleic acid inhibitor molecules. Such modifications can be made on any part of the nucleotide, including the sugar moiety, the phosphodiester linkage, and the nucleobase. Typical examples of nucleotide modification include, but are not limited to, 2′-F, 2′-O-methyl (“2′-OMe” or “2′-OCH₃”), and 2′-O-methoxyethyl (“2′-MOE” or “2′-OCH₂CH₂OCH₃”). Modifications can also occur at other parts of the sugar moiety of the nucleotide, such as the 5′-carbon, as described herein.

In certain embodiments, the double-stranded nucleic acid inhibitor molecule can also include one or more modified nucleobases other than adenine, guanine, cytosine, thymine and uracil at the 1′-position, as known in the art and as described herein. In certain embodiments, the modified or universal nucleobase is a nitrogenous base. In certain embodiments, the modified nucleobase does not contain a nitrogen atom. See e.g., U.S. Published Patent Application No. 20080274462. In certain embodiments, the modified nucleotide does not contain a nucleobase (abasic). A typical example of a modified nucleobase is 5′-methylcytosine.

The natural occurring internucleotide linkage of RNA and DNA is a 3′- to 5′-phosphodiester linkage. Modified phosphodiester linkages include non-naturally occurring internucleotide linking groups, including internucleotide linkages that contain a phosphorous atom and internucleotide linkages that do not contain a phosphorous atom, as known in the art and as described herein. Typically, the double-stranded nucleic acid inhibitor molecule contains one or more phosphorous-containing internucleotide linking groups, as described herein. In other embodiments, one or more of the internucleotide linking groups of the double-stranded nucleic acid inhibitor molecule is a non-phosphorus containing linkage, as described herein. In certain embodiments, the double-stranded nucleic acid inhibitor molecule contains one or more phosphorous-containing internucleotide linking groups and one or more non-phosphorous containing internucleotide linking groups.

In certain embodiments, the double-stranded nucleic acid inhibitor molecule contains at least one phosphorothioate internucleotide linking group. In certain embodiments, the double-stranded nucleic acid inhibitor molecule contains less than 10, such as less than 5 phosphorothioate internucleotide linking groups. In certain embodiments, the double-stranded nucleic acid inhibitor molecule contains 4 phosphorothioate internucleotide linking groups.

A 5′-end of the sense and/or antisense strand of the double-stranded nucleic acid inhibitor molecule can include a natural substituent, such as a hydroxyl or a phosphate group. In certain embodiments, a hydroxyl group is attached to the 5′-terminal end of the sense and/or antisense strand of the double-stranded nucleic acid inhibitor molecule. In certain embodiments, a phosphate group is attached to the 5′-terminal end of the sense and/or antisense strand of the double-stranded nucleic acid inhibitor molecule. Typically, the phosphate is added to a monomer prior to oligonucleotide synthesis. In other embodiments, 5′-phosphorylation is accomplished naturally after a nucleic acid inhibitor molecule is introduced into the cytosol, for example, by a cytosolic Clp1 kinase. In some embodiments, the 5′-terminal phosphate is a phosphate group, such as 5′-monophosphate [(HO)₂(O)P—O-5′], 5′-diphosphate [(HO)₂(O)P—O—P(HO)(O)—O-5′] or a 5′-triphosphate[(HO)₂(O)P—O—(HO)(O)P—O—P(HO)(O)—O-5′].

The 5′-end of the sense and/or antisense strand of the double-stranded nucleic acid inhibitor molecule can also be modified. For example, in some embodiments, the 5′-end of the sense and/or antisense strand of the double-stranded nucleic acid inhibitor molecule is attached to a phosphoramidate [(HO)₂(O)P—NH-5′, (HO)(NH₂)(O)P—O-5]. In certain embodiments, the 5′-terminal end of the sense and/or antisense strand of the double-stranded nucleic acid inhibitor molecule is attached to a phosphate mimic. Suitable phosphate mimics include 5′-phosphonates, such as 5′-methylenephosphonate (5′-MP), 5′-(E)-vinylphosphonate (5′-VP). Lima et al., Cell, 2012, 150-883-94; WO 2014/130607. Other suitable phosphate mimics include 4-phosphate analogs that are bound to the 4′-carbon of the sugar moiety (e.g., a ribose or deoxyribose or analog thereof) of the 5′-terminal nucleotide of an oligonucleotide as described in International Publication No. WO 2018/045317, which is hereby incorporated by reference in its entirety. For example, in some embodiments, the 5′-end of the sense and/or antisense strand of the double-stranded nucleic acid inhibitor molecule is attached to an oxymethylphosphonate, where the oxygen atom of the oxymethyl group is bound to the 4′-carbon of the sugar moiety or analog thereof. In other embodiments, the phosphate analog is a thiomethylphosphonate or an aminomethylphosphonate, where the sulfur atom of the thiomethyl group or the nitrogen atom of the aminomethyl group is bound to the 4′-carbon of the sugar moiety or analog thereof.

In certain embodiments, the double-stranded nucleic acid inhibitor molecule includes one or more deoxyribonucleotides. Typically, the double-stranded nucleic acid inhibitor molecules contain fewer than 5 deoxyribonucleotides. In certain embodiments, the double-stranded nucleic acid inhibitor molecules include one or more ribonucleotides. In certain embodiments, all of the nucleotides of the double-stranded nucleic acid inhibitor molecule are ribonucleotides.

In certain embodiments, one or more nucleotides outside of the stem (second duplex or D2) of the double-stranded nucleic acid inhibitor molecule contain a sugar moiety have a modified ring structure, including but not limited to, the modified ring structure present in bicyclic or tricyclic nucleotides, as described herein, and Unlocked Nucleic Acids (“UNA”) (see, e.g., Snead et al. (2013), Molecular Therapy—Nucleic Acids, 2,e103(doi: 10.1038/mtna.2013.36)).

In certain embodiments one or two nucleotides of the double-stranded nucleic acid inhibitor molecule are reversibly modified with a glutathione-sensitive moiety. Typically, the glutathione-sensitive moiety is located at the 2′-carbon of the sugar moiety and comprises a sulfonyl group. In certain embodiments, the glutathione-sensitive moiety is compatible with phosphoramidite oligonucleotide synthesis methods, as described in International Publication No. WO 2018/045317, which is hereby incorporated by reference in its entirety. In certain embodiments, more than two nucleotides of the double-stranded nucleic acid inhibitor molecule are reversibly modified with a glutathione-sensitive moiety. In certain embodiments, most of the nucleotides are reversibly modified with a glutathione-sensitive moiety. In certain embodiments, all or substantially all the nucleotides of the double-stranded nucleic acid inhibitor molecule are reversibly modified with a glutathione-sensitive moiety.

The at least one glutathione-sensitive moiety is typically located at the 5′- or 3′-terminal nucleotide of the sense strand or the antisense strand of the double-stranded nucleic acid inhibitor molecule. However, the at least one glutathione-sensitive moiety may be located at any nucleotide of interest in the double-stranded nucleic acid inhibitor molecule.

In certain embodiments, the double-stranded nucleic acid inhibitor molecule is fully modified, wherein every nucleotide of the sense strand and antisense strand is modified; typically every nucleotide is modified at the 2′-position of the sugar moiety. In certain embodiments, the fully modified nucleic acid inhibitor molecule does not contain a reversible modification. In some embodiments, at least one, such as at least two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, or 36 nucleotides of the sense strand of the double-stranded nucleic acid inhibitor molecule are modified. In some embodiments, at least one, such as at least two, three, four, five, six, seven, eight, nine, 10, 11, 12, 13, 14, 15, 16, 17, 18, 19, 20, 21, 22, 23, or 24 nucleotides of the antisense strand of the double-stranded nucleic acid inhibitor molecule are modified.

In certain embodiments, the fully modified nucleic acid inhibitor molecule is modified with one or more reversible, glutathione-sensitive moieties. In certain embodiments, substantially all of the nucleotides of the double-stranded nucleic acid inhibitor molecule are modified. In certain embodiments, more than half of the nucleotides of the double-stranded nucleic acid inhibitor molecule are modified with a chemical modification other than a reversible modification. In certain embodiments, less than half of the nucleotides of the double-stranded nucleic acid inhibitor molecule are modified with a chemical modification other than a reversible modification. Modifications can occur in groups on the nucleic acid inhibitor molecule or different modified nucleotides can be interspersed.

In certain embodiments of the double-stranded nucleic acid inhibitor molecule, from one to every nucleotide is modified at the 2′-carbon. In certain embodiments, the double-stranded nucleic acid inhibitor molecule is partially or fully modified with 2′-F, 2′-OMe, and/or 2′-MOE. In certain embodiments of the double-stranded nucleic acid inhibitor molecule, from one to every phosphorous atom is modified and from one to every nucleotide is modified at the 2′-carbon of the sugar moiety.

In certain embodiments of the double-stranded nucleic acid inhibitor molecule, every nucleotide on the sense and antisense strands is modified at the 2′-carbon of the sugar moiety. In certain embodiments of the double-stranded nucleic acid inhibitor molecule, every nucleotide on the sense and antisense strands is modified at the 2′-carbon of the sugar moiety with a 2′-F or a 2′-OMe, except for the nucleotides in the second region of the sense strand (R2). In certain embodiments of the double-stranded nucleic acid inhibitor molecule, every nucleotide on the sense and antisense strands is modified at the 2′-carbon of the sugar moiety with a 2′-F or a 2′-OMe, except for the Tₘ-increasing nucleotides in the stem (second duplex or D2). In certain embodiments of the double-stranded nucleic acid inhibitor molecule, every nucleotide on the sense and antisense strands is modified at the 2′-carbon of the sugar moiety with a 2′-F or a 2′-OMe, except for the Tₘ-increasing nucleotides in the stem (second duplex or D2) and the nucleotides in the triloop that are conjugated to a ligand moiety, such as GalNAc.

Methods of Reducing Target Gene Expression

The triloop-containing double-stranded nucleic acid inhibitor molecule, as described herein, can be used in methods of reducing target mRNA expression of any target gene of interest. Typically, the method of reducing mRNA expression comprises administering the double-stranded nucleic acid inhibitor molecule, as described herein, to a sample or to a subject in need thereof in an amount sufficient to reduce mRNA expression of the target gene. The methods may be carried out in vitro or in vivo.

The level or activity of a target RNA can be determined by a suitable method now known in the art or that is later developed. It can be appreciated that the method used to measure a target RNA and/or the “expression” of a target gene can depend upon the nature of the target gene and its encoded RNA. For example, where the target RNA sequence encodes a protein, the term “expression” can refer to a protein or the target RNA/transcript derived from the target gene (either genomic or of exogenous origin). In such instances the expression of the target RNA can be determined by measuring the amount of target RNA/transcript directly or by measuring the amount of protein encoded by the target RNA/transcript. Protein can be measured in protein assays such as by staining or immunoblotting or, if the protein catalyzes a reaction that can be measured, by measuring reaction rates. All such methods are known in the art and can be used. Where target RNA levels are to be measured, art-recognized methods for detecting RNA levels can be used (e.g., RT-PCR, Northern Blotting, etc.). The above measurements can be made on cells, cell extracts, tissues, tissue extracts or another suitable source material.

Pharmaceutical Compositions

The present disclosure provides pharmaceutical compositions comprising a therapeutically effective amount of the triloop-containing double-stranded nucleic acid inhibitor molecule, as described herein, and a pharmaceutically acceptable excipient.

These pharmaceutical compositions may be sterilized by conventional sterilization techniques, or may be sterile filtered. The resulting aqueous solutions may be packaged for use as is, or lyophilized, the lyophilized preparation being combined with a sterile aqueous excipient prior to administration. The pH of the preparations typically will be between 3 and 11, more preferably between 5 and 9 or between 6 and 8, and most preferably between 7 and 8, such as 7 to 7.5.

The pharmaceutical compositions of the present disclosure are applied for therapeutic use. Thus, one aspect of the disclosure provides a pharmaceutical composition, which may be used to treat a subject including, but not limited to, a human suffering from a disease or a condition by administering to said subject a therapeutically effective amount of a pharmaceutical composition of the present disclosure. In certain embodiments, the disease or condition is cancer, as described herein.

In certain embodiments, the present disclosure features the use of a therapeutically effective amount of a pharmaceutical composition as described herein for the manufacture of a medicament for treatment of a subject in need thereof. In certain embodiments, the subject has cancer, as described herein.

Pharmaceutically-Acceptable Excipients

The pharmaceutically-acceptable excipients useful in this disclosure are typically conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; malt; gelatin; excipients, such as cocoa butter and suppository waxes; oils, such as peanut oil, cottonseed oil, safflower oil, sesame oil, olive oil, corn oil and soybean oil; buffering agents, such as magnesium hydroxide and aluminum hydroxide; (isotonic saline; Ringer's solution); ethyl alcohol; pH buffered solutions; polyols, such as glycerol, propylene glycol, polyethylene glycol, and the like; and other non-toxic compatible substances employed in pharmaceutical formulations.

Dosage Forms

The pharmaceutical compositions may be formulated with conventional excipients for any intended route of administration, which may be selected according to ordinary practice.

In one embodiment, the pharmaceutical composition contains the triloop-containing double-stranded nucleic acid inhibitor molecule, as described herein, and is suitable for parenteral administration, for example, by subcutaneous, intramuscular, intravenous or epidural injection. Typically, the pharmaceutical compositions of the present disclosure are formulated in liquid form for parenteral administration.

Dosage forms suitable for parenteral administration typically include one or more suitable vehicles for parenteral administration including, by way of example, sterile aqueous solutions, saline, low molecular weight alcohols such as propylene glycol, polyethylene glycol, vegetable oils, gelatin, fatty acid esters such as ethyl oleate, and the like. The parenteral formulations may contain sugars, alcohols, antioxidants, buffers, bacteriostats, solutes which render the formulation isotonic with the blood of the intended recipient or suspending or thickening agents. Proper fluidity can be maintained, for example, by the use of surfactants. Liquid formulations containing the double-stranded nucleic acid inhibitor can be lyophilized and stored for later use upon reconstitution with a sterile injectable solution.

The pharmaceutical compositions may also be formulated for other routes of administration including topical or transdermal administration, rectal or vaginal administration, ocular administration, nasal administration, buccal administration, or sublingual administration using well known techniques.

Delivery Agents

The triloop-containing double-stranded nucleic acid inhibitor molecule, as described herein, may be admixed, encapsulated, conjugated or otherwise associated with other molecules, molecule structures or mixtures of compounds, including, for example, liposomes and lipids such as those disclosed in U.S. Pat. Nos. 6,815,432, 6,586,410, 6,858,225, 7,811,602, 7,244,448 and 8,158,601; polymeric materials such as those disclosed in U.S. Pat. Nos. 6,835,393, 7,374,778, 7,737,108, 7,718,193, 8,137,695 and U.S. Published Patent Application Nos. 2011/0143434, 2011/0129921, 2011/0123636, 2011/0143435, 2011/0142951, 2012/0021514, 2011/0281934, 2011/0286957 and 2008/0152661; capsids, capsoids, or receptor targeted molecules for assisting in uptake, distribution or absorption.

In certain embodiments, the triloop-containing double-stranded nucleic acid inhibitor molecule is formulated in a lipid nanoparticle (LNP). Lipid-nucleic acid nanoparticles typically form spontaneously upon mixing lipids with nucleic acid to form a complex. Depending on the desired particle size distribution, the resultant nanoparticle mixture can be optionally extruded through a polycarbonate membrane (e.g., 100 nm cut-off) using, for example, a thermobarrel extruder, such as LIPEX® Extruder (Northern Lipids, Inc). To prepare a lipid nanoparticle for therapeutic use, it may desirable to remove solvent (e.g., ethanol) used to form the nanoparticle and/or exchange buffer, which can be accomplished by, for example, dialysis or tangential flow filtration. Methods of making lipid nanoparticles containing nucleic acid interference molecules are known in the art, as disclosed, for example in U.S. Published Patent Application Nos. 2015/0374842 and 2014/0107178.

In certain embodiments, the LNP comprises a core lipid component comprising a cationic liposome and a pegylated lipid. The LNP can further comprise one or more envelope lipids, such as a cationic lipid, a structural or neutral lipid, a sterol, a pegylated lipid, or mixtures thereof.

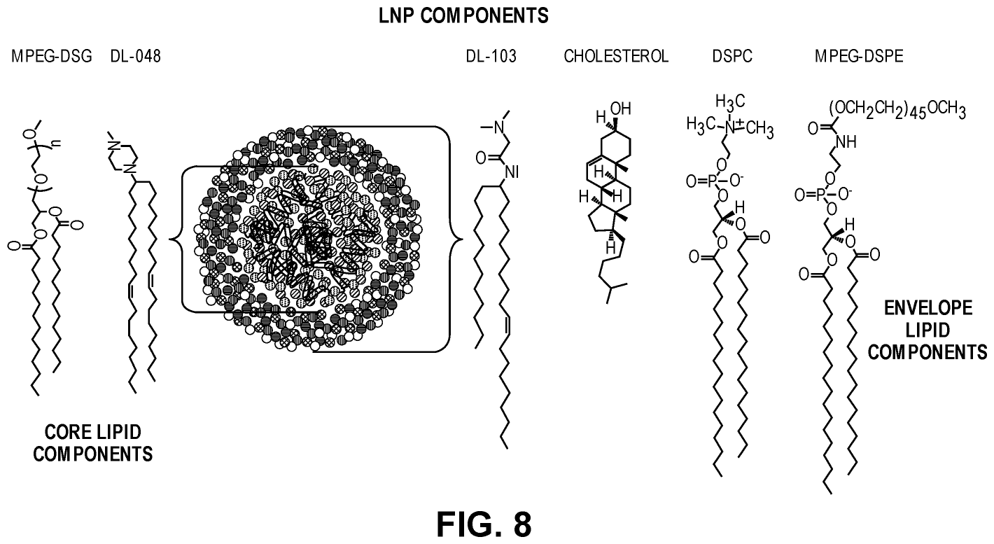

Cationic lipids for use in LNPs are known in the art, as discussed for example in U.S. Published Patent Application Nos. 2015/0374842 and 2014/0107178. Typically, the cationic lipid is a lipid having a net positive charge at physiological pH. In certain embodiments, the cationic liposome is DODMA, DOTMA, DL-048, or DL-103. In certain embodiments the structural or neutral lipid is DSPC, DPPC or DOPC. In certain embodiments, the sterol is cholesterol. In certain embodiments, the pegylated lipid is DMPE-PEG, DSPE-PEG, DSG-PEG, DMPE-PEG2K, DSPE-PEG2K, DSG-PEG2K, or DSG-mPEG. In one embodiment, the cationic lipid is DL-048, the pegylated lipid is DSG-mPEG and the one or more envelope lipids are DL-103, DSPC, cholesterol, and DSPE-mPEG. See e.g., FIG. 8, showing one non-limiting embodiment of a LNP that can used to formulate the double-stranded nucleic acid inhibitor molecule.

In certain embodiments, the triloop-containing double-stranded nucleic acid inhibitor molecule is covalently conjugated to a ligand that directs delivery of the oligonucleotide to a tissue of interest. Many such ligands have been explored. See, e.g., Winkler, Ther. Deliv. 4(7): 791-809 (2013). For example, the double-stranded nucleic acid inhibitor molecule can be conjugated to one or more sugar ligand moieties (e.g., N-acetylgalactosamine (GalNAc)) to direct uptake of the oligonucleotide into the liver. See, e.g., U.S. Pat. Nos. 5,994,517; 5,574,142; WO 2016/100401. In certain embodiments, the one or more ligands are conjugated to one or more nucleotides in the triloop of the double-stranded nucleic acid inhibitor molecule.

In certain embodiments, the double-stranded nucleic acid inhibitor molecule is conjugated to two or three sugar ligand moieties in the triloop. In one embodiment, two of the nucleotides in the triloop are conjugated to a sugar ligand moiety. In another embodiment, three of the nucleotides in the triloop are conjugated to a sugar ligand moiety. In certain embodiments, the sugar ligand moiety is GalNAc. In one embodiment, the sugar ligand moiety is GalNAc and is conjugated to two of the nucleotides in the triloop. In one embodiment, GalNAc is conjugated to the AA nucleotides of the GAA triloop. Other ligands that can be used include, but are not limited to, mannose-6-phosphate, cholesterol, folate, transferrin, and galactose (for other specific exemplary ligands see, e.g., WO 2012/089352).

The ligand can be conjugated to any part of the nucleotide as long as it is capable of directing delivery of the oligonucleotide to the tissue of interest. In certain embodiments, the ligand (e.g., GalNAc) is conjugated to the nucleotide at the 2′-position of the sugar moiety.

Methods of Administration/Treatment

One embodiment is directed to a method of treating a disorder, comprising administering to a subject a pharmaceutical composition comprising a therapeutically effective amount of the triloop-containing double-stranded nucleic acid inhibitor molecule, as described herein.

In certain embodiments the pharmaceutical compositions disclosed herein may be useful for treatment or prevention of symptoms related to proliferative, inflammatory, autoimmune, neurologic, ocular, respiratory, metabolic, dermatological, auditory, liver, kidney, or infectious diseases. One embodiment is directed to a method of treating a proliferative, inflammatory, autoimmune, neurologic, ocular, respiratory, metabolic, dermatological, auditory, liver, kidney, or infectious disease, comprising administering to a subject a pharmaceutical composition comprising a therapeutically effective amount of a double-stranded nucleic acid inhibitor molecule, as described herein.

In certain embodiments, the disorder is a rare disease, a chronic liver disease, a chronic kidney disease, cardiovascular disease or a viral infectious disease. In certain embodiments, the disorder is hyperoxaluria, including primary hyperoxaluria (PH1, PH2, or PH3) or idiopathic hyperoxaluria. In certain embodiments, the disorder is chronic kidney disorder (CKD). In certain embodiments, the disorder is pyruvate dehydrogenase deficiency. In certain embodiments, the disorder is alpha-I antitrypsin (A1AT) deficiency.

In certain embodiments, the disorder is a cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, wilms tumor, leukemia, acute lymocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T-cell lymphomas, non-Hodgkin lymphoma, precursor T-lymphoblastic lymphoma/leukemia, peripheral T-cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Typically, the present disclosure features methods of treating liver cancer, liver carcinoma, hepatoma, hepatocellular carcinoma, cholangiocarcinoma and hepatoblastoma by administering a therapeutically effective amount of a pharmaceutical composition as described herein.

In some embodiments, the present disclosure provides a method for reducing expression of a target gene in a subject comprising administering a pharmaceutical composition to a subject in need thereof in an amount sufficient to reduce expression of the target gene, wherein the pharmaceutical composition comprises a triloop-containing double-stranded nucleic acid inhibitor molecule as described herein and a pharmaceutically acceptable excipient as also described herein.

The target gene may be a target gene from any mammal, such as a human target gene. Any target gene may be silenced according to the instant methods. In certain embodiments, the target gene is associated with chronic liver disease or chronic kidney disease, including, for example, AGXT, GRHPR, HOGA1, HAO1, SERPINA1, or LDHA. In certain embodiments, the target gene is associated with a viral infectious disease, including, for example, an HBV gene or an HCV gene. In certain embodiments, the target gene is associated with cardiovascular disease, including, for example, APOC3 or PCSK9. In certain embodiments, the target gene is associated with alcohol metabolism and liver function, including, for example, ALDH2.

Other exemplary target genes include, but are not limited to, KRAS, Factor VII, EgS, PCSK9, TPX2, apoB, SAA1, TTR, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, p73 gene, p21(WAF1/CIP1) gene, p27(KIP1) gene, PPM1D gene, RAS gene, caveolin I gene, MIB I gene, MTAI gene, M68 gene, mutations in tumor suppressor genes, p53 tumor suppressor gene, and combinations thereof

Dosing and Schedule

Typically, the double-stranded nucleic acid inhibitor molecule is administered parenterally (such as via intravenous, intramuscular, or subcutaneous administration). In other embodiments, the pharmaceutical composition is delivered via local administration or systemic administration. However, the pharmaceutical compositions disclosed herein may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In certain embodiments, the double-stranded nucleic acid inhibitor molecule is administered at a dosage of 20 micrograms to 10 milligrams per kilogram body weight of the recipient per day, 100 micrograms to 5 milligrams per kilogram, 0.1 milligrams to 5.0 milligrams per kilogram, 0.25 milligrams to 5.0 milligrams per kilogram, or 0.2 milligrams to 3.0 milligrams per kilogram. Typically, the double-stranded nucleic acid inhibitor molecule is administered at a dosage of about 0.25 milligrams to 2.0 milligrams per kilogram body weight of the recipient per day, such as 0.3 milligrams per kilogram body weight of the recipient per day, 0.5 milligrams per kilogram body weight of the recipient per day, or 1 milligram per kilogram body weight of the recipient per day.

A pharmaceutical composition of the instant disclosure may be administered every day, or intermittently. For example, intermittent administration of the double-stranded nucleic acid inhibitor molecule may be administration one to six days per week, one to six days per month, once weekly, once every other week, once monthly, once every other month, once every three months, or once or twice per year or divided into multiple yearly, monthly, weekly, or daily doses. In some embodiments, intermittent dosing may mean administration in cycles with the initial double-stranded nucleic acid inhibitor molecule administration followed by a rest period with no administration for up to one week, up to one month, up to two months, up to three months or up to six months or more) or it may mean administration on alternate days, weeks, months or years.

The therapeutically effective amount of the double-stranded nucleic acid inhibitor molecule may depend on the route of administration and the physical characteristics of the patient, such as the size and weight of the subject, the extent of the disease progression or penetration, the age, health, and sex of the subject and can be adjusted as necessary depending on these and other factors.

### EXAMPLES

### Example 1: In Vivo Dose Response of Double-Stranded Nucleic Acid Inhibitor Molecules Containing Triloops and Tetraloops

Double-stranded nucleic acid inhibitor molecules containing tetraloops and triloops in addition to varying stem lengths were evaluated in a dose-response study. CD-1 female mice were divided into study groups and dosed with the test nucleic acid inhibitor molecule assigned to that group. Four CD-1 female mice were dosed with 0.1 mg/kg, 0.2 mg/kg, 0.4 mg/kg, 0.8 mg/kg, 1.6 mg/kg, and 3.2 mg/kg of each of the five tested nucleic acid inhibitor molecules, respectively. Additionally, four control CD-1 mice were dosed with a placebo (PBS), such that the total sample size was 124 mice. Dosing was subcutaneous and single dose, and the mice were sacrificed 4 days post-dose. A pharmacodynamics study was conducted, and liver samples were taken for RT-qPCR. Tissue samples were homogenized in QIAzol® Lysis Reagent using TissueLyser II (Qiagen, Valencia, Calif.). RNA was then purified using MagMAX Technology according to manufacturer instructions (ThermoFisher Scientific, Waltham, Mass.). High capacity cDNA reverse transcription kit (ThermoFisher Scientific, Waltham, Mass.) was used to prepare cDNA. Primers for the target sequence were used for PCR on a CFX384 Real-Time PCR Detection System (Bio-Rad Laboratories, Inc., Hercules, Calif.).

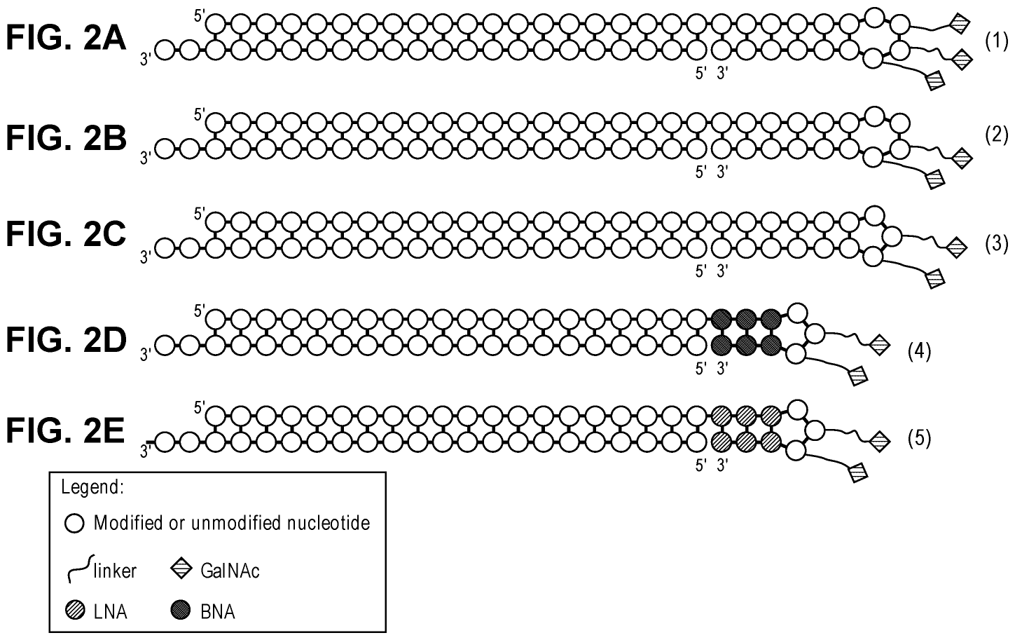

The five test nucleic acid inhibitor molecules used in Example 1 (Constructs 1-5) are shown in FIGS. 2A-E. With the exception of the nucleotides in the loop that are conjugated to GalNAc and the bicyclic nucleotides, every other nucleotide in the test nucleic acid inhibitor molecules is modified at the 2′-position of the sugar moiety with either 2′-F or 2′-OMe. The test nucleic acid inhibitor molecules differed in the following respects: length of the loop portion (tetraloop vs. triloop); length of the stem portion (6 base pairs vs. 3 base pairs); presence or absence of bicyclic nucleotides; and number of GalNAcs in loop (2 vs. 3). The nucleic acid inhibitor molecules in FIGS. 2A-E are summarized in the following table:

TABLE 1
Test nucleic acid inhibitors in Figure 1

| | | Loop | | Bicyclic | GaINAcs |
|---|---|---|---|---|---|
| FIG. | Name | Length | Stem Length | nucleotides | in loop |
| 2A | Construct 1 | 4 | Long (6 base pairs) | None | 3 |
| 2B | Construct 2 | 4 | Long (6 base pairs) | None | 2 |
| 2C | Construct 3 | 3 | Long (6 base pairs) | None | 2 |
| 2D | Construct 4 | 3 | Short (3 base pairs) | BNA^{NC} | 2 |
| | | | | [NMe] | |
| 2E | Construct 5 | 3 | Short (3 base pairs) | LNA | 2 |

The long stem (6 base pair) tetraloop Constructs 1 and 2 are identical with the only difference being the number of GalNAcs in the loop (3 vs. 2). The long stem Construct 2 is identical to the long stem Construct 3 except that Construct 2 has a tetraloop, whereas Construct 3 has a triloop. The long stem Construct 3 is identical to the short stem (3 base pairs) Construct 4 except that Construct 3 has a stem duplex of 6 base pairs and no bicyclic nucleotides, whereas construct 4 has a stem duplex of 3 base pairs, wherein all of the nucleotides in the stem duplex are bicyclic nucleotides. The short stem Constructs 4 and 5 are identical with the only difference being the presence of 6 BNA^{NC} [NMe] in the stem of Construct 4 and the presence of 6 LNA bicyclic nucleotides in the stem of Construct 5. The bicyclic nucleotide used in Construct 4 is BNA^{NC} [NMe], where the bridge that connects the 2′-carbon and the 4′-carbon of the bicyclic nucleotide is 4′-CH₂—N(CH₃)—O-2′. The bicyclic nucleotide used in the Construct 5 is a LNA, wherein the bridge that connects the 2′-carbon and the 4′-carbon of the bicyclic nucleotide is 4′-(CH₂)—O-2′.

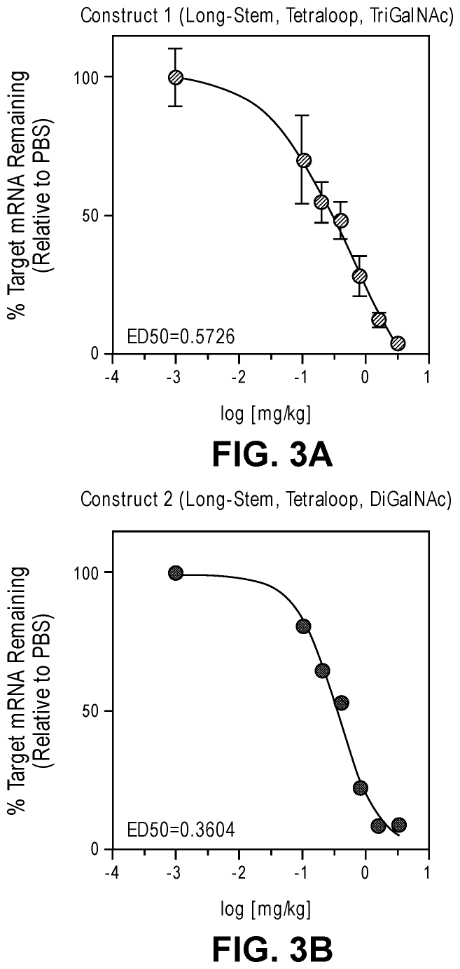

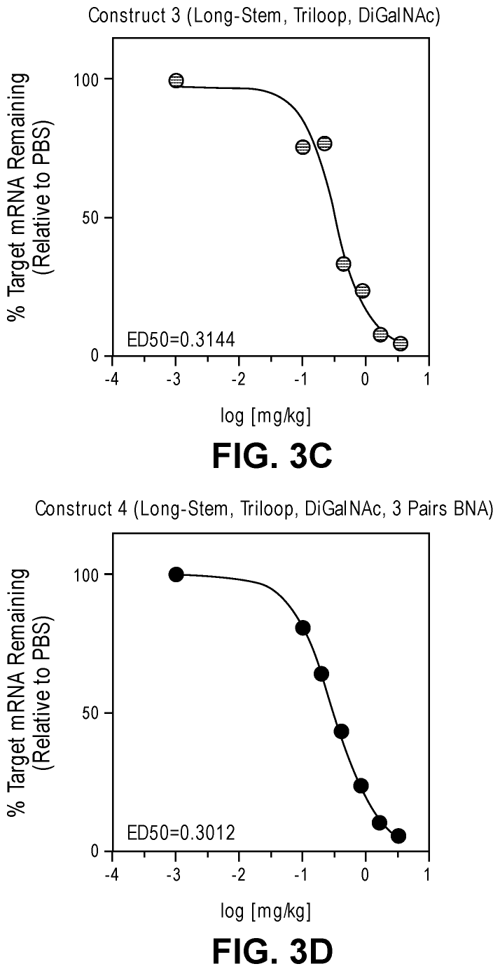

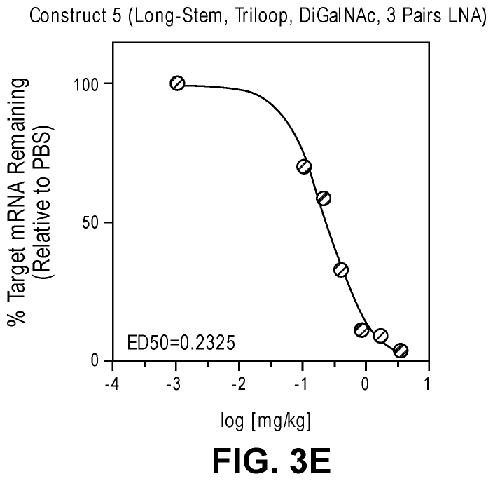

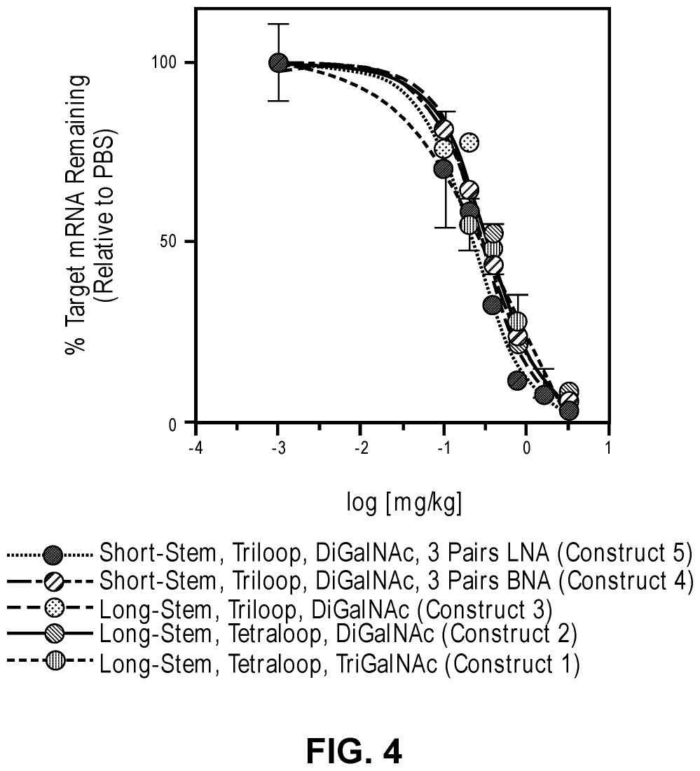

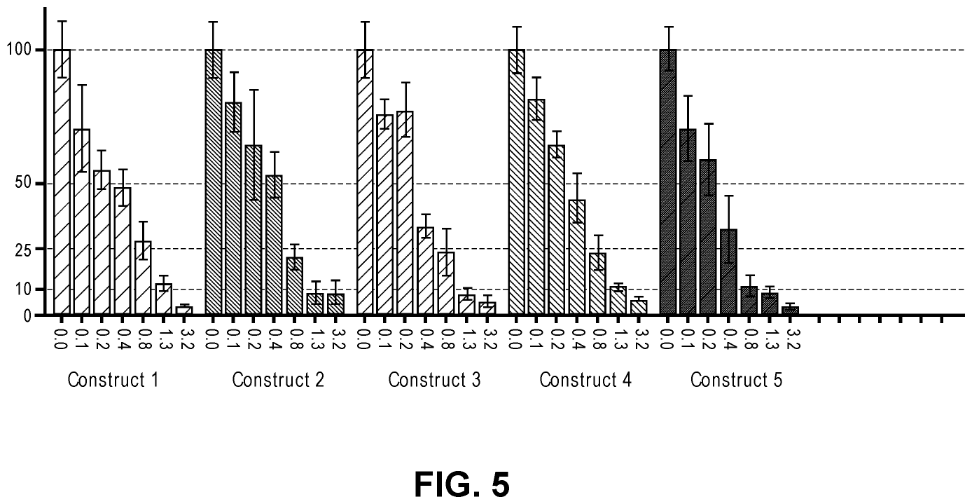

Effective Dose (ED₅₀) curves were calculated based on the 50% percent target gene remaining relative to PBS over log [mg/kg], using nonlinear regression analysis (GraphPad Prism software). The tetraloop long stem (6 base pairs) nucleic acid molecules were compared to the triloop long stem nucleic acid molecules. Additionally, the triloop short stem (3 base pairs) nucleic acid inhibitor molecules containing BNA^{NC} [NMe] were compared to the corresponding triloop short stem nucleic acid molecules containing LNA. They were also compared to the triloop and tetraloop longer stem (6 base pairs in the stem) nucleic acid molecules without the bicyclic nucleotides. As seen in FIGS. 3 and 4, both the triloop longer stem nucleic acid inhibitor molecule (Construct 3) and the triloop shorter stem nucleic acid molecules containing bicyclic nucleotides in the short stem (Constructs 4 and 5) showed similar potency of the target gene knock down, as compared to the corresponding tetraloop nucleic acid molecules containing either 2 or 3 GalNAcs (Constructs 1 and 2).

### Example 2: In Vivo Potency of Double-Stranded Nucleic Acid Inhibitor Molecules Containing Tetraloops and Triloops for Target Gene Knockdown

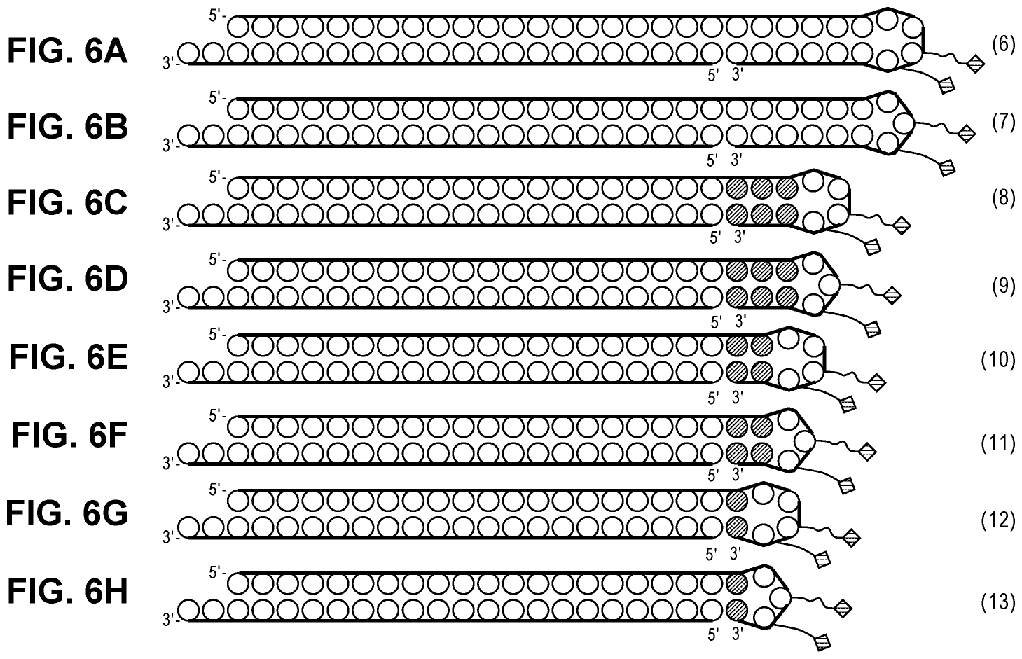

CD-1 female mice were divided into study groups and dosed with test nucleic acid inhibitor molecule assigned to that group. The test nucleic acid inhibitor molecules used in Example 2 (Constructs 6-13) are shown in FIGS. 6A-H. Construct 1 (see FIG. 2A) was also used. With the exception of the nucleotides in the loop that are conjugated to GalNAc and the bicyclic nucleotides, every other nucleotide in the test nucleic acid inhibitor molecules is modified at the 2′-position of the sugar moiety with either 2′-OMe or 2′-F. The test nucleic acid inhibitor molecules differed in the following respects: length of the loop portion (tetraloop vs. triloop); length of the stem portion (6 base pairs, 3 base pairs, 2 base pairs, and 1 base pair); and presence or absence of bicyclic nucleotides. The nucleic acid inhibitor molecules in FIGS. 6A-H are summarized in the following table:

TABLE 2
Test nucleic acid inhibitors in Figure 6

| | | Loop | | Bicyclic |
|---|---|---|---|---|
| FIG. | Name | Length | Stem Length | nucleotides |
| 6A | Construct 6 | 4 | 6 base pairs | None |
| 6B | Construct 7 | 3 | 6 base pairs | None |
| 6C | Construct 8 | 4 | 3 base pairs | BNA |
| 6D | Construct 9 | 3 | 3 base pairs | BNA |
| 6E | Construct 10 | 4 | 2 base pairs | BNA |
| 6F | Construct 11 | 3 | 2 base pairs | BNA |
| 6G | Construct 12 | 4 | 1 base pair | BNA |
| 6H | Construct 13 | 3 | 1 base pair | BNA |

The long stem (6 base pairs) Constructs 6 and 7 are identical with the only difference being the number of nucleotides in the loop (tetraloop vs. triloop). Construct 1 is identical to Construct 6 except that Construct 1 contains 3 GalNAcs conjugated to the tetraloop, whereas Construct 6 contains 2 GalNAcs conjugated to the tetraloop. Construct 8 (3 base pair stem) is identical to Construct 9 (3 base pair stem) except that Construct 8 has a tetraloop, whereas Construct 9 has a triloop. Constructs 10 and 11 (2 base pair stem) are identical with the only difference being Construct 10 has a tetraloop, whereas construct 11 has a triloop. Constructs 12 and 13 (1 base pair stem) are identical with the only difference being Construct 12 has a tetraloop, whereas Construct 13 has a triloop. The bicyclic nucleotides used in Constructs 8-13 are BNA^{NC} [NMe].

Animals were dosed subcutaneously with a single 0.5 mg/kg does of the assigned test nucleic acid inhibitor molecule, and the mice were sacrificed 4 days post-dose. Liver tissue was collected by taking two 4 mm punch biopsies which were stored in Invitrogen™ RNAlater™ solution (Thermo Fisher Scientific, Waltham, Mass.) for later mRNA analysis. Tissue samples were homogenized in QIAzol® Lysis Reagent using TissueLyser II (Qiagen, Valencia, Calif.). RNA was then purified using MagMAX Technology according to manufacturer instructions (ThermoFisher Scientific, Waltham, Mass.). High capacity cDNA reverse transcription kit (ThermoFisher Scientific, Waltham, Mass.) was used to prepare cDNA. Primers for the target sequence were used for PCR on a CFX384 Real-Time PCR Detection System (Bio-Rad Laboratories, Inc., Hercules, Calif.).

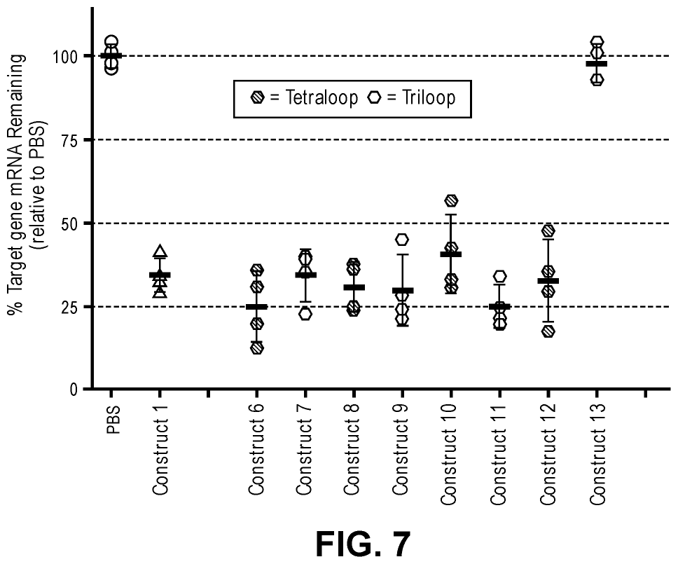

The triloop test nucleic acid inhibitor molecules (Constructs 7, 9, 11, and 13) were compared to the corresponding tetraloop version of the test nucleic acid molecules (Constructs 1, 6, 8, 10, and 12, respectively). As demonstrated in FIG. 7, the test nucleic acid inhibitor molecules containing triloops showed similar knockdown of the target gene mRNA as compared to the corresponding tetraloop test nucleic acid molecules for the 6 base pair, 3 base pair, and 2 base pair constructs. Construct 11, containing a triloop conjugated to 2 GalNAcs and having 2 base pairs in the stem, had higher potency than Construct 1, containing a triloop conjugated to 3 GalNAcs and 6 base pairs in the stem. Construct 13, containing a triloop with 1 base pair in the stem, did not exhibit knockdown of the target gene mRNA, whereas Construct 12, containing a tetraloop and 1 base pair in the stem, showed substantial knockdown of target gene mRNA.

1. A double-stranded nucleic acid inhibitor molecule, comprising:
a sense strand comprising 20-65 nucleotides and having a first region (R1) and a second region (R2);
an antisense strand comprising 15-40 nucleotides, wherein the sense strand and antisense strand are separate strands; and
a first duplex (D1) formed by the first region of the sense strand and the antisense strand, wherein the first duplex has a length of 15-40 base pairs;
wherein the second region of the sense strand comprises a first subregion (S1), a second subregion (S2) and a triloop (triL) that joins the first and second regions, wherein the first and second regions form a second duplex (D2).

2. The double-stranded nucleic acid inhibitor molecule of claim 1, wherein the triloop has a nucleotide sequence of GAA.

3. The double-stranded nucleic acid inhibitor molecule of claim 1, wherein the sense strand has 22-65, 25-39, or 27-35 nucleotides.

4. The double-stranded nucleic acid inhibitor molecule of claim 1, wherein the antisense strand has 20-24 or 20-22 nucleotides.

5. The double-stranded nucleic acid inhibitor molecule of claim 1, wherein the nucleotide immediately adjacent to the 5′-end of the triloop is a C and the nucleotide immediately adjacent to the 3′-end of the triloop is a G.

6. The double-stranded nucleic acid inhibitor molecule of claim 1, wherein the antisense strand has a single stranded overhang of 1-4 nucleotides at its 3′-end.

7. The double-stranded nucleic acid inhibitor molecule of claim 6, wherein the single stranded overhang is 2 nucleotides in length.

8. The double-stranded nucleic acid inhibitor molecule of claim 1, wherein the first duplex has a length of 18-30, 18-24, or 20-22 base pairs.

9. The double-stranded nucleic acid inhibitor molecule of claim 1, wherein the second duplex has a length of 2-6 base pairs.

10. The double-stranded nucleic acid inhibitor molecule of claim 1, wherein the second duplex comprises at least one Tₘ-increasing nucleotide, such as a bicyclic nucleotide.

11. The double-stranded nucleic acid inhibitor molecule of claim 1, wherein the second duplex has a length of 2 or 3 base pairs.

12. The double-stranded nucleic acid inhibitor molecule of claim 1, wherein the first region of the sense strand is 20 nucleotides in length and the second region of the sense strand is 7-15 nucleotides in length;
wherein the first duplex formed by the first region of the sense strand and the antisense strand has a length of 20 base pairs;
wherein the second duplex formed by the first and second nucleic acids of the second region of the sense strand has a length of 2-6 base pairs; and
wherein the antisense strand is 22 nucleotides in length and has a single-stranded overhang of two nucleotides at its 3′-end.

13. The double-stranded nucleic acid inhibitor molecule of claim 1, wherein each nucleotide in the second duplex is a Tₘ-increasing nucleotide.

14. The double-stranded nucleic acid inhibitor molecule of claim 13, wherein the second duplex has a length of 6 base pairs.

15. The double-stranded nucleic acid inhibitor molecule of claim 1, wherein the second duplex comprises at least one Tₘ-increasing nucleotide is a bicyclic nucleotide chosen from one or more of the following: 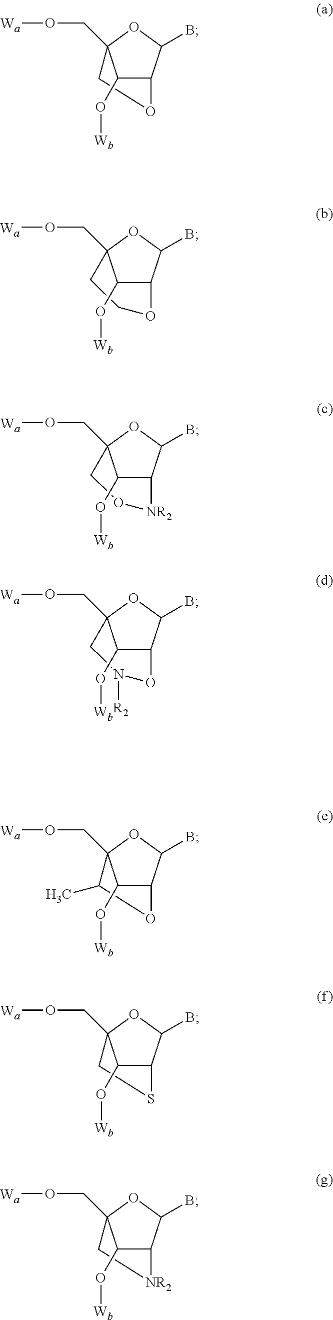 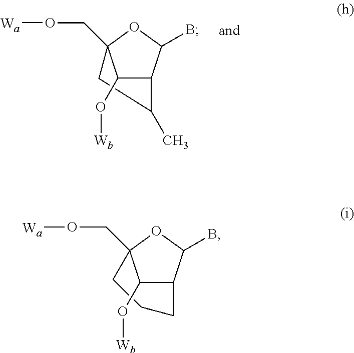
wherein B is a nucleobase, R₂ is H or CH₃ and Wₐ and W_{b} are each independently, H, OH,
a hydroxyl protecting group, a phosphorous moiety, or an internucleotide linking group attaching the bicyclic nucleotide to another nucleotide or to an oligonucleotide and wherein at least one of Wₐ or W_{b} is an internucleotide linking group attaching the bicyclic nucleotide to an oligonucleotide.

16. The double-stranded nucleic acid inhibitor molecule of claim 1, wherein the second duplex comprises at least one bicyclic nucleotide that comprises a first ring, wherein the first ring is a furanosyl, and a bridge that connects the 2′-carbon and the 4′-carbon of the furanosyl to form a second ring.

17. The double-stranded nucleic acid inhibitor molecule of claim 16, wherein the bridge that connects the 2′-carbon and the 4′-carbon of the furanosyl is selected from the group consisting of:
a) 4′-CH₂—O—N(R)-2′ and 4′-CH₂—N(R)—O-2′, wherein R is H, C₁-C₁₂ alkyl, or a protecting group, including, for example, 4′-CH₂—NH—O-2′ (also known as BNA^{NC}) or 4′-CH₂—N(CH₃)—O-2′ (also known as BNA^{NC}[NMe]);
b) 4′-CH₂-2; 4′—(CH₂)₂-2; 4′—(CH₂)₃-2; 4′—(CH₂)—O-2′ (also known as LNA); 4′-(CH₂)—S-2; 4′—(CH₂)₂—O-2′ (also known as ENA); 4′-CH(CH₃)—O-2′ (also known as cEt); and 4′-CH(CH₂OCH₃)—O-2′ (also known as cMOE), and analogs thereof;
c) 4′-C(CH₃)(CH₃)—O-2′ and analogs thereof;
d) 4′-CH₂—N(OCH₃)-2′ and analogs thereof;
e) 4′-CH₂—O—N(CH₃)-2′ and analogs thereof;
f) 4′-CH₂—C(H)(CH₃)-2′ and analogs thereof; and
g) 4′-CH₂—C(═CH₂)-2′ and analogs thereof.

18. The double-stranded nucleic acid inhibitor molecule of claim 1, wherein the triloop comprises at least one ligand conjugated nucleotide.

19. The double-stranded nucleic acid inhibitor molecule of claim 1, wherein the triloop comprises at least two ligand conjugated nucleotides.

20. The double-stranded nucleic acid inhibitor molecule of claim 18, wherein the ligand is a GalNAc.

21. The double-stranded nucleic acid inhibitor molecule of claim 20, wherein the GalNAc is conjugated to the nucleotide at the 2′-position of the sugar moiety.

22. The double-stranded nucleic acid inhibitor molecule of claim 1, further comprising a 5′-phosphate mimic at the 5′-terminus of the sense strand and/or the antisense strand.

23. The double-stranded nucleic acid inhibitor molecule of claim 1, wherein the double stranded the nucleic acid inhibitor molecule is formulated with a lipid nanoparticle.

24. The double-stranded nucleic acid inhibitor molecule of claim 23, wherein the lipid nanoparticle comprises core lipids and envelope lipids, wherein the core lipids comprise a first cationic lipid and a first pegylated lipid and wherein the envelope lipids comprise a second cationic lipid, a neutral lipid, a sterol, and a second pegylated lipid.

25. The double-stranded nucleic acid inhibitor molecule of claim 24, wherein the first cationic lipid is DL-048, the first pegylated lipid is DSG-MPEG, the second cationic lipid is DL-103, the neutral lipid is DSPC, the sterol is cholesterol, and the second pegylated lipid is DSPE-MPEG.

26. A pharmaceutical composition comprising a therapeutically effective amount of the double-stranded nucleic acid inhibitor molecule of claim 1 and a pharmaceutically acceptable excipient.

27. A method for reducing expression of a target gene in a subject comprising administering the double-stranded nucleic acid inhibitor molecule or pharmaceutical composition of claim 1 to a subject in need thereof in an amount sufficient to reduce expression of the target gene.

28. The method of claim 27, wherein the administering comprises intravenous, intramuscular, or subcutaneous administration.

29. The method of claim 27, wherein the subject is a human.
